(19) United States
(12) Patent Application Publication
(10) Pub. No.: US 2023/0390210 A1
(43) Pub. Date: Dec. 7, 2023

(54) MODIFIED RELEASE SOFTGEL CAPSULES

(71) Applicant: R.P. SCHERER TECHNOLOGIES, LLC, Carson City, NV (US)

(72) Inventors: Karunakar Sukuru, St. Petersburg, FL (US); Qi Fang, St. Petersburg, FL (US)

(21) Appl. No.: 18/031,669

(22) PCT Filed: Oct. 15, 2021
(86) PCT No.: PCT/US2021/071900

§ 371 Date: Apr. 13, 2023

Related U.S. Application Data
(60) Provisional application No. 63/092,762, filed on Oct. 16, 2020.

(51) Int. Cl. A61K 9/48; A61K 47/36

(52) U.S. Cl. CPC A61K 9/4866; A61K 9/4825; A61K 47/36

(57) ABSTRACT

Described are modified release softgel capsules including a pH dependent shell composition that encapsulated a controlled release fill composition, methods of preparation thereof, and methods of use thereof. The pH dependent shell composition may be characterized in that the delayed release nature of the capsules may be achieved without a separate pH dependent coating or added conventional pH dependent polymers. The softgels provide a dual controlled release platform which facilitates delivery of the active agent to a target location in the gastrointestinal tract and a controlled release profile of the active agent at said target location in the gastrointestinal tract.

### FIELD OF THE DISCLOSURE

The present disclosure relates to modified release softgel capsules that encapsulate a controlled release fill composition. The pH dependent shell compositions of the gelatin-based capsules possess delayed release properties without the need for a separate pH dependent coatings or the addition of conventional pH dependent synthetic polymers. The controlled release fill compositions possess controlled release properties. Together, the pH dependent shell composition and controlled release fill composition allow delivery of an active agent to a target location within the gastrointestinal tract and allow tuning the release profile of the active agent in said target location.

### BACKGROUND OF THE DISCLOSURE

Soft capsules, in particular, soft gelatin capsules (or softgel capsules), provide a dosage form which is more readily accepted by patients, since the capsules are easy to swallow and need not be flavored in order to mask any unpleasant taste of the active agent.

Efforts have been made to create delayed release dosage forms. Delayed release dosage forms are designed to protect the contents of the dosage forms from gastric conditions. For example, delayed release dosage forms may be produced by adding a pH dependent coating to the surface of a manufactured dosage form such as a tablet or a capsule. Such coatings may be applied through spraying the dosage form, followed by drying the dosage form, usually at elevated temperatures. This method of coating a capsule with a pH dependent coating may lead to disadvantages in terms of performance and appearance. For example, the capsule may appear rough, the coating may be applied unevenly, and/or the coating can be prone to cracking or flaking off the dosage form. Additionally, the process of applying a pH dependent coating is very inefficient.

Other delayed release dosage forms have been developed in which conventional pH dependent polymers (i.e., acid-insoluble polymers) are added in the capsule shell. However, the addition of conventional pH dependent polymers can lead to capsules that are prone to leaking due to insufficient sealing.

Accordingly, there is currently a need for a modified release softgel capsule that does not require either an application of a separate pH dependent coating or the addition of conventional pH dependent polymers in the shell.

Softgel capsules could also benefit from the ability to tune and/or control the release profile of the active agent from the fill composition of the softgel capsule after the shell composition ruptures/dissolves/disintegrates.

### SUMMARY OF THE DISCLOSURE

The present disclosure is directed to modified release softgel capsules, such as dual controlled release softgel capsules. The modified release softgel capsules comprise (a) a controlled release fill composition and (2) a pH dependent shell composition encapsulating the controlled release fill composition. In an embodiment, the pH dependent shell composition comprises gelatin. In an embodiment, the pH dependent shell composition comprises a pH dependent release material (e.g., pectin). In an embodiment, the pH dependent shell composition comprises dextrose. In an embodiment, the pH dependent shell composition comprises a plasticizer (e.g., glycerol, sorbitol, or a combination thereof). In an embodiment, the pH dependent shell composition comprises a binder (e.g., gellan gum). In an embodiment, the pH dependent shell composition comprises a combination of two or more of gelatin, pectin, dextrose, plasticizer, and a binder.

In some embodiments, the modified release softgel capsule does not require a separate pH dependent coating (e.g., over the pH dependent shell composition). Accordingly, the pH dependent shell composition included in the modified release softgel capsule eliminates, in some embodiments, the need to add a separate pH dependent coating, which also minimizes the risk of damaging the capsules during the coating process.

In an embodiment, the pH dependent shell composition comprises: (a) a gelatin, (b) dextrose, (c) a pectin such as a low methoxyl pectin and optionally (d) a plasticizer. The pH dependent shell composition (e.g., amount of pectin, amount of dextrose, gelatin to pectin ratio) and its preparation process (e.g., curing duration, ribbon thickness) may be tuned/adjusted/modified to attain a target pH dissolution profile of the shell composition at various pH environments (e.g., rupture/dissolution/disintegration time in acidic medium and in buffer medium).

The controlled release fill composition includes at least one active agent and a controlled release material. The active agent may be a pharmaceutically active ingredient or a nutraceutical. The controlled release material may be a polyethylene oxide, a cellulose derivative, a gum, or a combination thereof. In certain embodiments, the controlled release fill composition further includes a hydrophilic carrier, such as a polyol (e.g., polyethylene glycol, polypropylene glycol), or water.

In an embodiment, the controlled release fill composition (e.g., type and amount of active agent, type and amount of controlled release material, and optionally type and amount of the hydrophilic carrier, as well as the ratios between these materials) and its preparation process (e.g., annealing duration) may be tuned/adjusted/modified to attain a target release profile of the active agent from the controlled release fill composition (e.g., zero order release). In certain embodiments, the modified release softgel capsule may be annealed. In an embodiment, an annealed modified release softgel capsule comprises the controlled release fill composition in a form of a matrix (solid or liquid) of the controlled release material encapsulated in the pH dependent shell composition.

In certain embodiments, disclosed is a modified release softgel capsule including a controlled release fill composition, including: (i) at least one active agent, (ii) a polyethylene oxide having a number average molecular weight of from about 0.05M Dalton to about 15M Dalton; and (iii) optionally a hydrophilic carrier; and a pH dependent shell composition encapsulating the controlled release fill composition, wherein the pH dependent shell composition comprises gelatin, pectin, dextrose, and optionally a plasticizer.

The modified release softgel capsules described herein may also be referred to as dual controlled release softgel capsules due to the two levels of controlled release that they possess. The first level of controlled release being due to the pH dependent shell composition of the softgel capsule. The second level of controlled release being due to the controlled release fill composition of the softgel capsule.

The present disclosure is also directed to a process of preparing modified release softgel capsules. In certain embodiments, disclosed is a process of preparing a modified release softgel capsule which includes: mixing at least one active agent with polyethylene oxide and optionally a hydrophilic carrier to form a controlled release fill composition; encapsulating the controlled release fill composition in a pH dependent shell composition comprising gelatin, pectin, dextrose, and optionally a plasticizer; and annealing the encapsulated controlled release fill composition.

In certain embodiments, the present disclosure is also directed to a method of tuning each level of the dual controlled release mechanism of the softgel capsule to facilitate targeted release of the active agent to specific areas within the gastrointestinal tract with a targeted active agent release profile. For instance, in certain embodiments, the modified release softgel capsules described herein deliver the active agent to a lower portion of the gastrointestinal tract (e.g., close to the colon) and release the active agent in a controlled manner (e.g., with a zero order release for a duration of about 2 hours to about 24 hours).

The present disclosure is also directed to a method of treating a condition by administering to a subject any of the delayed release softgel compositions described herein.

### BRIEF DESCRIPTION OF THE DRAWINGS

The above and other features of the present disclosure, their nature, and various advantages will become more apparent upon consideration of the following detailed description, taken in conjunction with the accompanying drawings, in which:

FIG. 1 illustrates viscosity of shell compositions with amidated pectin and non-amidated pectin as a function of aging time.

FIG. 2 illustrates the release profile of ibuprofen from modified release softgel capsules, according to embodiments described herein, having varying concentration of a controlled release material in the controlled release fill composition.

FIG. 3 illustrates the release profile of diphenhydramine from a modified release softgel capsule, according to an embodiment.

FIG. 4 illustrates the release profile of acetaminophen from a modified release softgel capsule, according to an embodiment.

FIG. 5 depicts an exemplary process for preparing a modified release softgel capsule, according to an embodiment.

FIG. 6 depicts the dissolution profiles of capsules, according to embodiments, obtained in a fiberoptic dissolution test using USP Apparatus II using a paddle speed of 100 RPM at 37° C. in 500 ml of water run at 100 RPM.

FIG. 7 depicts the dissolution profiles of capsules, according to embodiments, obtained in a fiberoptic dissolution test using USP Apparatus II using a paddle speed of 50 RPM at 37° C. in 500 ml of water.

FIGS. 8A-8D and 9-10 show the residual plots for time 90% (hours) for the statistical analysis of dissolution data of Examples 13-18.

FIG. 8A is a normal probability plot for time to release 90% (hours).

FIG. 8B is a versus fits plot for time to release 90% (hours).

FIG. 8C is a histogram for time to release 90% (hours).

FIG. 8D is a versus order plot for time to release 90% (hours).

FIG. 9 is an interaction plot for time to release 90% (hours).

FIG. 10 is a main effects plot for time to release 90% (hours).

FIG. 11 depicts the dissolution profiles for capsules, according to embodiments, filled with formulations 13-15 obtained in a fiberoptic dissolution test using USP Apparatus II using a paddle speed of 100 RPM at 37° C. in 500 ml of water.

FIG. 12 depicts the dissolution profiles for capsules, according to embodiments, obtained in a fiberoptic dissolution test using USP Apparatus II using a paddle speed of 100 RPM at 37° C. in 500 ml of water.

FIG. 13 depicts the dissolution profiles for capsules, according to embodiments, obtained in a fiberoptic dissolution test using USP Apparatus II using a paddle speed of 50 RPM at 37° C. in 500 ml of water.

FIG. 14 is a DSC curve of heat flow vs temperature for a capsule fill composition containing polyethylene oxide having a number average molecular weight of 900,000 Da.

FIG. 15 is a DSC curve of heat flow vs temperature for a capsule fill composition containing the MC18-30 fill mix.

FIG. 16 is a DSC curve of heat flow vs temperature for a capsule fill composition containing polyethylene oxide having a number average molecular weight of 5,000,000 Da.

FIG. 17 is a DSC curve of heat flow vs temperature for a capsule fill composition containing the MC18-31 fill mix.

FIG. 18 is a DSC curve of heat flow vs temperature for a capsule fill composition containing polyethylene oxide having a number average molecular weight of 7,000,000 Da.

FIG. 19 is a DSC curve of heat flow vs temperature for a capsule fill composition containing the MC18-32 fill mix.

### DETAILED DESCRIPTION OF THE DISCLOSURE

The present disclosure advances the state of the art by developing dual controlled release oral dosage forms, in particular, modified release softgel capsules with two levels of controlled release. The first level being attributed to a pH dependent shell composition that achieves the advantages associated with the conventional delayed release dosage forms without the need to apply a pH dependent coating or to add conventional pH dependent synthetic polymer in the capsule shell. The second level being attributed to the controlled release fill composition.

The pH dependent shell composition of the modified release softgel capsules described herein do not dissolve in a gastric environment of the stomach, but rather dissolve in a pH that is about 3.5 or above (e.g., in the duodenal area and/or in the intestines). The dissolution profile of the pH dependent shell compositions described herein can be tuned by modifying the constituents and method of preparation of the shell composition.

The controlled release fill composition of the modified release softgel capsules described herein can be tuned by modifying the constituents and method of preparation of the fill composition. Among other factors, the controlled release nature of the fill composition may be tuned by incorporation of a controlled release material (such as polyethylene oxide, cellulose derivative, gums, or a combination of two or more thereof).

The dual controlled release mechanism of the modified release softgel capsules described herein is beneficial for delivery of active agents that may cause stomach irritation or bleeding (like NSAIDs) or are sensitive to the acidic environment of the stomach (like peptides and proteins/enzymes). Such mechanism is also beneficial for reducing belching after consuming capsules that encapsulate fill compositions that tend to contribute to belching (like fish oil, garlic oil, or krill oil). For instance, belching often occurs upon consuming vitamin, minerals, supplements, and/or pharmaceutical products that are formulated in dosage form exhibiting some leaking (even of a very small amount), in the stomach, before reaching the intestines. The leakage can be particularly problematic when the belching is associated with substances that have a noisome perception such as fish oil and garlic oil that are commonly delivered in softgels. The modified release softgel capsules described herein may be formulated in a manner that minimizes and/or eliminates premature leakage (and consequently premature release of the capsule's fill) in the gastric environment of the stomach. The modified release softgel capsules described herein can also be utilized to deliver active agents (e.g., peptides and proteins) to the lower portion of the gastrointestinal tract and/or close to the colon area where certain active agents are absorbed better.

### Definitions

As used herein, the term “pH dependent” is used to refer to the dissolution or disintegration resistant property of a substance such that dissolution or disintegration does not occur or does not substantially occur in a gastric environment of the stomach, e.g., for a time period of at least about 15 minutes, at least about 30 minutes, at least about one hour, at least about two hours, at least about three hours, at least about four hours, or at least about five hours. In certain embodiments, the gastric environment of the stomach may be simulated here with 0.1N HCl and optionally with the addition of pepsin. It should be noted that pharmacopeial methods do not include pepsin, however, pepsin was added in certain dissolution/disintegration tests described herein to better simulate/mimic in-vivo conditions. Hence, without being construed as limited, in certain embodiments, the compositions described herein are resistant to dissolution/disintegration for the durations outlined above even at 0.1N HCl environments that include Pepsin (which is presumed to be a more aggressive environment than 0.1N HCl without Pepsin).

For example, the embodiments described herein include a pH dependent shell composition that preferentially dissolves in pH of about 3.5 or higher (e.g., in biological, artificial or simulated duodenal environment and/or intestinal fluid) as compared to biological, artificial or simulated gastric fluid. In certain embodiments, the intestinal environment may be simulated here with pH 6.8 phosphate buffer with or without Pancreatin. For instance, pH dependent shell compositions described herein dissolve in pH of about 3.5 or higher (e.g., in biological, artificial or simulated duodenal environment and/or intestinal fluid such as pH 6.8 phosphate buffer optionally with Pancreatin) in less than about 60 minutes, less than about 45 minutes, less than about 30 minutes, less than about 20 minutes, less than about 10 minutes, or less than about 5 minutes. It should be noted that pharmacopeial methods do not include pancreatin, however, pancreatin was added in certain dissolution/disintegration tests described herein to better simulate/mimic in-vivo conditions. Hence, without being construed as limited, in certain embodiments, the compositions described herein exhibit similar dissolution/disintegration profiles at pH 6.8 buffer environments that include Pancreatin (which is presumed to be a more aggressive environment than pH 6.8 buffer environment without Pancreatin).

As used herein, “pharmaceutically active ingredient,” “active agents” refers to a drug or compound that may be used in the diagnosis, cure, mitigation, treatment, or prevention of a condition. In certain embodiments, suitable “active agents” include nutraceuticals, such as, vitamins, minerals, and supplements (VMS). Exemplary modified release softgel capsules may include, without limitations, capsules containing, in the fill composition, lactic acid bacteria, probiotics, fish oil, krill oil, valproic acid, garlic oil, peppermint oil, nonsteroidal anti-inflammatory drugs (NSAIDs) (e.g., ibuprofen solution or suspension), proton pump inhibitors, aspirin, antihistamines (e.g., diphenhydramine), acetaminophen, drugs that are susceptible to abuse (e.g., opioids), drugs that are not susceptible to abuse, and similar products.

The term “condition” or “conditions” refers to those medical conditions that can be treated or prevented by administration to a subject of an effective amount of an active agent.

As used herein, the term “active ingredient” refers to any material that is intended to produce a therapeutic, prophylactic, or other intended effect, whether or not approved by a government agency for that purpose. This term with respect to a specific agent includes the pharmaceutically active agent, and all pharmaceutically acceptable salts, solvates and crystalline forms thereof, where the salts, solvates and crystalline forms are pharmaceutically active.

Any pharmaceutically active ingredient may be used for purposes of the present disclosure, including both those that are water-soluble and those that are poorly soluble in water. Suitable pharmaceutically active ingredients include, without limitation, analgesics and anti-inflammatory agents (e.g., ibuprofen, naproxen sodium, aspirin), antacids, anthelmintic, anti-arrhythmic agents, anti-bacterial agents, anti-coagulants, anti-depressants, anti-diabetics, anti-diarrheal, anti-epileptics, anti-fungal agents, anti-gout agents, anti-hypertensive agents, anti-malarial, anti-migraine agents, anti-muscarinic agents, anti-neoplastic agents and immunosuppressants, anti-protozoal agents, anti-rheumatics, anti-thyroid agents, anti-histamines (e.g., diphenhydramine), antivirals, anxiolytics, sedatives, hypnotics and neuroleptics, beta-blockers, cardiac inotropic agents, corticosteroids, cough suppressants, cytotoxics, decongestants, diuretics, enzymes, anti-parkinsonian agents, gastro-intestinal agents, histamine receptor antagonists, lipid regulating agents, local anesthetics, neuromuscular agents, nitrates and anti-anginal agents, nutritional agents, opioid analgesics, anticonvulsant agents (e.g., valporic acid), oral vaccines, proteins, peptides and recombinant drugs, sex hormones and contraceptives, spermicides, stimulants, and combinations thereof.

In some embodiments, the active pharmaceutical ingredient may be selected, without limitations, from the group consisting of dabigatran, dronedarone, ticagrelor, iloperidone, ivacaftor, midostaurine, asimadoline, beclomethasone, apremilast, sapacitabine, linsitinib, abiraterone, vitamin D analogs (e.g., calcifediol, calcitriol, paricalcitol, doxercalciferol), COX-2 inhibitors (e.g., celecoxib, valdecoxib, rofecoxib), tacrolimus, testosterone, lubiprostone, pharmaceutically acceptable salts thereof, and combinations thereof.

In some embodiments, the lipids in the dosage form may be selected, without limitations, from the group consisting of almond oil, argan oil, avocado oil, borage seed oil, canola oil, cashew oil, castor oil, hydrogenated castor oil, cocoa butter, coconut oil, colza oil, corn oil, cottonseed oil, grape seed oil, hazelnut oil, hemp oil, hydroxylated lecithin, lecithin, linseed oil, macadamia oil, mango butter, manila oil, mongongo nut oil, olive oil, palm kernel oil, palm oil, peanut oil, pecan oil, perilla oil, pine nut oil, pistachio oil, poppy seed oil, pumpkin seed oil, peppermint oil, rice bran oil, safflower oil, sesame oil, shea butter, soybean oil, sunflower oil, hydrogenated vegetable oil, walnut oil, and watermelon seed oil. Other oil and fats may include, but not be limited to, fish oil (omega-3), krill oil, garlic oil, animal or vegetable fats, e.g., in their hydrogenated form, free fatty acids and mono-, di-, and tri-glycerides with C8-, C10-, C12-, C14-, C16-, C18-, C20- and C22-fatly acids, fatty acid esters like EPA and DHA 3 and combinations thereof.

According to certain embodiments, active agents may include lipid-lowering agents including, but not limited to, statins (e.g., lovastatin, simvastatin, pravastatin, fluvastatin, atorvastatin, rosuvastatin, and pitavastatin), fibrates (e. g, clofibrate, ciprofibrate, bezafibrate, fenofibrate, and gemfibrozil), niacin, bile acid sequestrants, ezetimibe, lomitapide, phytosterols, and the pharmaceutically acceptable salts, hydrates, solvates and prodrugs thereof, mixtures of any of the foregoing, and the like.

Suitable nutraceutical active agents may include, but are not limited to, 5-hydroxytryptophan, acetyl L-carnitine, alpha lipoic acid, alpha-ketoglutarates, bee products, betaine hydrochloride, bovine cartilage, caffeine, cetyl myristoleate, charcoal, chitosan, choline, chondroitin sulfate, coenzyme Q10, collagen, colostrum, creatine, cyanocobalamin (Vitamin 812), dimethylaminoethanol, fumaric acid, germanium sequioxide, glandular products, glucosamine HCl, glucosamine sulfate, hydroxyl methyl butyrate, immunoglobulin, lactic acid, L-Carnitine, liver products, malic acid, maltose-anhydrous, mannose (d-mannose), methyl sulfonyl methane, phytosterols, picolinic acid, pyruvate, red yeast extract, S-adenosylmethionine, selenium yeast, shark cartilage, theobromine, vanadyl sulfate, and yeast.

Suitable nutritional supplement active agents may include vitamins, minerals, fiber, fatty acids, amino acids, herbal supplements or a combination thereof.

Suitable vitamin active agents may include, but are not limited to, the following: ascorbic acid (Vitamin C), B vitamins, biotin, fat soluble vitamins, folic acid, hydroxycitric acid, inositol, mineral ascorbates, mixed tocopherols, niacin (Vitamin B3), orotic acid, para-aminobenzoic acid, panthothenates, panthothenic acid (Vitamin B5), pyridoxine hydrochloride (Vitamin B6), riboflavin (Vitamin B2), synthetic vitamins, thiamine (Vitamin B1), tocotrienols, vitamin A, vitamin D, vitamin E, vitamin F, vitamin K, vitamin oils and oil soluble vitamins.

Suitable herbal supplement active agents may include, but are not limited to, the following: arnica, bilberry, black cohosh, cat's claw, chamomile, echinacea, evening primrose oil, fenugreek, flaxseed, feverfew, garlic oil, ginger root, ginko biloba, ginseng, goldenrod, hawthorn, kava-kava, licorice, milk thistle, psyllium, rauowolfia, senna, soybean, St. John's wort, saw palmetto, turmeric, valerian.

Minerals active agents may include, but are not limited to, the following: boron, calcium, chelated minerals, chloride, chromium, coated minerals, cobalt, copper, dolomite, iodine, iron, magnesium, manganese, mineral premixes, mineral products, molybdenum, phosphorus, potassium, selenium, sodium, vanadium, malic acid, pyruvate, zinc and other minerals.

Examples of other possible active agents include, but are not limited to, antihistamines (e.g., ranitidine, dimenhydrinate, diphenhydramine, chlorpheniramine and dexchlorpheniramine maleate), non-steroidal anti-inflammatory agents (e.g., aspirin, celecoxib, Cox-2 inhibitors, diclofenac, benoxaprofen, flurbiprofen, fenoprofen, flubufen, indoprofen, piroprofen, carprofen, oxaprozin, pramoprofen, muroprofen, trioxaprofen, suprofen, aminoprofen, fluprofen, bucloxic acid, indomethacin, sulindac, zomepirac, tiopinac, zidometacin, acemetacin, fentiazac, clidanac, oxpinac, meclofenamic acid, flufenamic acid, niflumic acid, tolfenamic acid, diflurisal, flufenisal, piroxicam, sudoxicam, isoxicam, aceclofenac, aloxiprin, azapropazone, benorilate, bromfenac, carprofen, choline magnesium salicylate, diflunisal, etodolac, etoricoxib, faislamine, fenbufen, fenoprofen, flurbiprofen, ibuprofen, indometacin, ketoprofen, ketorolac, lomoxicam, loxoprofen, meloxicam, mefenamic acid, metamizole, methyl salicylate, magnesium salicylate, nabumetone, naproxen, nimesulide, oxyphenbutazone, parecoxib, phenylbutazone, salicyl salicylate, sulindac, sulfinpyrazone, tenoxicam, tiaprofenic acid, tolmetin. pharmaceutically acceptable salts thereof and mixtures thereof) and acetaminophen, anti-emetics (e.g., metoclopramide, methylnaltrexone), anti-epileptics (e.g., phenyloin, meprobmate and nitrazepam), vasodilators (e.g., nifedipine, papaverine, diltiazem and nicardipine), anti-tussive agents and expectorants (e.g. codeine phosphate), anti-asthmatics (e.g. theophylline), antacids, anti-spasmodics (e.g. atropine, scopolamine), antidiabetics (e.g., insulin), diuretics (e.g., ethacrynic acid, bendrofluthiazide), anti-hypotensives (e.g., propranolol, clonidine), antihypertensives (e.g., clonidine, methyldopa), bronchodilatiors (e.g., albuterol), steroids (e.g., hydrocortisone, triamcinolone, prednisone), antibiotics (e.g., tetracycline), antihemorrhoidals, hypnotics, psychotropics, antidiarrheals, mucolytics, sedatives, decongestants (e.g. pseudoephedrine), laxatives, vitamins, stimulants (including appetite suppressants such as phenylpropanolamine) and cannabinoids, as well as pharmaceutically acceptable salts, hydrates, solvates, and prodrugs thereof.

The active agent that may also be a benzodiazepine, barbiturate, stimulants, or mixtures thereof. The term “benzodiazepines” refers to a benzodiazepine and drugs that are derivatives of a benzodiazepine that are able to depress the central nervous system. Benzodiazepines include, but are not limited to, alprazolam, bromazepam, chlordiazepoxide, clorazepate, diazepam, estazolam, flurazepam, halazepam, ketazolam, lorazepam, nitrazepam, oxazepam, prazepam, quazepam, temazepam, triazolam, as well as pharmaceutically acceptable salts, hydrates, solvates, prodrugs and mixtures thereof. Benzodiazepine antagonists that can be used as active agent include, but are not limited to, flumazenil as well as pharmaceutically acceptable salts, hydrates, solvates and mixtures thereof.

The term “barbiturates” refers to sedative-hypnotic drugs derived from barbituric acid (2, 4, 6,-trioxohexahydropyrimidine). Barbiturates include, but are not limited to, amobarbital, aprobarbotal, butabarbital, butalbital, methohexital, mephobarbital, metharbital, pentobarbital, phenobarbital, secobarbital as well as pharmaceutically acceptable salts, hydrates, solvates, prodrugs, and mixtures thereof. Barbiturate antagonists that can be used as active agent include, but are not limited to, amphetamines as well as pharmaceutically acceptable salts, hydrates, solvates and mixtures thereof.

The term “stimulants” includes, but is not limited to, amphetamines such as dextroamphetamine resin complex, dextroamphetamine, methamphetamine, methylphenidate, as well as pharmaceutically acceptable salts, hydrates, and solvates and mixtures thereof. Stimulant antagonists that can be used as active agent include, but are not limited to, benzodiazepines, as well as pharmaceutically acceptable salts, hydrates, solvates and mixtures thereof.

In one embodiment of the present invention, the active pharmaceutical ingredient is a pain medication such as ibuprofen or an opioid. The term “opioid” refers to a psychoactive compound that works by binding to opioid receptors. Opioids are commonly used in the medical field for their analgesic effects. Opioids are believed to be APIs susceptible to abuse. Examples of opioids include codeine, tramadol, anileridine, prodine, pethidine, hydrocodone, morphine, oxycodone, methadone, diamorphine, hydromorphone, oxymorphone, 7-hydroxymitragynine, buprenorphine, fentanyl, sufentanil, levorphanol, meperidine, tilidine, dihydrocodeine, dihydromorphine, and pharmaceutically acceptable salts thereof.

Additional examples of the active pharmaceutical ingredient may include N-{1-[2-(4-ethyl-5-oxo-2-tetrazolin-1-yl)ethyl]-4-methoxymethyl-4-piperidyl}propionanilide; alfentanil; 5,5-diallylbarbituric acid; allobarbital; allylprodine; alphaprodine; 8-chloro-1-methyl-6-phenyl-4H-[1,2,4]triazolo[4,3-a][1,4]-benzodiazepine; alprazolam; 2-diethylaminopropiophenone; amfepramone, (±)-αmethylphenethylamine; amphetamine; 2-(α-methylphenethylamino)-2-phenylacetonitrile; amphetaminil; 5-ethyl-5-isopentylbarbituric acid; amobarbital; anileridine; apocodeine; 5,5-diethylbarbituric acid; barbital; benzylmorphine; bezitramide; 7-bromo-5-(2-pyridyl)-1H-1,4-benzodiazepine-2(3H)-one; bromazepam; 2-bromo-4-(2-chlorophenyl)-9-methyl-1-6H-thieno[3,2-f][1,2,4]triazolo[4,3-a][1,4]diazepine; brotizolam, 17-cyclopropylmethyl-4,5a-epoxy-7a[(S)-1-hydroxy-1,2,2-trimethyl-propyl]-6-methoxy-6,14-endo-ethanomorphinan-3-ol; buprenorphine; 5-butyl-5-ethylbarbituric acid; butobarbital; butorphanol; (7-chloro-1,3-dihydro-1-methyl-2-oxo-5-phenyl-2H-1,4-benzodiazepin-3-yl)dimethylcarbamate; camazepam; (1S,2S)-2-amino-1-phenyl-1-propanol; cathine; d-norpseudoephedrine; 7-chloro-N-methyl-5-phenyl-3H-1,4-benzodiazepin-2-yl-amine 4-oxide; chlordiazepoxide, 7-chloro-1-methyl-5-phenyl-1H-1,5-benzodiazepine-2,4(3H,5H)-dione; clobazam, 5-(2-chlorophenyl)-7-nitro-1H-1,4-benzodiazepin-2(3H)-one; clonazepam; clonitazene; 7-chloro-2,3-dihydro-2-oxo-5-phenyl-1H-1,4-benzodiazepine-3-carboxylic acid; clorazepate; 5-(2-chlorophenyl)-7-ethyl-1-methyl-1H-thieno[2,3-e][1,4]diazepin-2(3H)-one; clotiazepam; 10-chloro-11b-(2-chlorophenyl)-2,3,7,11b-tetrahydrooxazol-o [3,2-d][1,4]benzodiazepin-6(5H)-one; cloxazolam; (−)-methyl-[3β-benzoyloxy-2β(1αH,5αH)-tropane carboxylate]; cocaine; (5α,6α)-7,8-didehydro-4,5-epoxy-3-methoxy-17-methylmorphinan-6-ol; 4,5α-epoxy-3-methoxy-17-methyl-7-morphinen-6α-ol; codeine; 5-(1-cyclohexenyl)-5-ethyl barbituric acid; cyclobarbital; cyclorphan; cyprenorphine; 7-chloro-5-(2-chloropheny-1)-1H-1,4-benzodiazepin-2(3H)-one; delorazepam; desomorphine; dextromoramide; (+)-(1-benzyl-3-dimethyl amino-2-methyl-1-phenylpropyl)propionate; dextropropoxyphene; dezocine; diampromide; diamorphone; 7-chloro-1-methyl-5-phenyl-1H-1,4-benzodiazepin-2(3H)-on; diazepam; 4,5α-epoxy-3-methoxy-17-methyl-6α-morphinanol; dihydrocodeine; 4,5α-epoxy-17-methyl-3,6a-morphinandiol; dihydromorphine; dimenoxadol; dimephetamol; dimethylthiambutene; dioxaphetyl butyrate; dipipanone; (6aR, 10aR)-6,6,9-trimethyl-3-pentyl-6a,7,8,10a-tetrahy dro-6H-benzo[c]chromen-1-ol; dronabinol; eptazocine; 8-chloro-6-phenyl-4H-[1,2,4]-triazolo[4,3-(a)][1,4]benzodiazepine; estazolam; ethoheptazine; ethylmethylthiambutene; ethyl[7-chloro-5-(2-fluorophenyl)-2,3-dihydro-2-oxo-1H-1,4-benzodiazepine-3-carboxylate]; ethyl loflazepate; 4,5α-epoxy-3-ethoxy-17-methyl-7-morphinen-6α-ol; ethylmorphine; etonitazene; 4,5α-epoxy-7α-(1-hydroxy-1-methylbutyl)-6-methoxy-17-methyl-6,14-endo-etheno-morphinan-3-ol; etorphine; N-ethyl-3-phenyl-8,9,10-trinorbornan-2-ylamine; fencamfamine; 7-[2-(α-methylphenethylamino)ethyl]-theophylline; fenethylline; 3-(α-methylphenethylamino)propionitrile; fenproporex; N-(1-phenethyl-4-piperidyl)propionanilide; fentanyl; 7-chloro-5-(2-fluorophenyl)-1-methyl-1H-1,4-benzodiazepin-2(3H)-one; fludiazepam; 5-(2-fluorophenyl)-1-methyl-7-nitro-1H-1,4-benzodiazepin-2(3H)-one; flunitrazepam; 7-chloro-1-(2-diethylaminoethyl)-5-(2-fluorophenyl)-1H-1,4-benzodiazepin-2(3H)-one; flurazepam; 7-chloro-5-phenyl-1-(2,2,2-trifluoroethyl)-1H-1,4-benzodiazepin-2(3H)-one; halazepam; 10-bromo-11b-(2-fluorophenyl)-2,3,7,11b-tetrahydro[1,3]oxazolyl[3,2-d][1,4]benzodiazepin-6(5H)-one; haloxazolam; heroin; 4,5α-epoxy-3-methoxy-17-methyl-6-morphinanone; hydrocodone; 4,5α-epoxy-3-hydroxy-17-methyl-6-morphinanone; hydromorphone; hydroxypethidine; isomethadone; hydroxymethylmorphinan; 11-chloro-8,12b-dihydro-2,8-dimethyl-12b-phenyl-4H-[1,3]oxazino[3,2d][1,4]benzodiazepine-4,7(6H)-dione; ketazolam; 1-[4-(3-hydroxyphenyl)-1-methyl-4-piperidyl]-1-propanone; ketobemidone; (3S,6S)-6-dimethylamino-4,4-diphenylheptan-3-yl acetate; levacetylmethadol; LAAM; (−)-6-dimethylamino-4,4-diphenol-3-heptanone; levomethadone; (−)-17-methyl-3-morphinanol; levorphanol; levophenacylmorphane; lofentanil; 6-(2-chlorophenyl)-2-(4-methyl-1-piperazinylmethylene)-8-nitro-2H-imidazo[1,2-a][1,4]-benzodiazepin-1(4H)-one; loprazolam; 7-chloro-5-(2-chlorophenyl)-3-hydroxy-1H-1,4-benzodiazepin-2(3H)-one; lorazepam; 7-chloro-5-(2-chlorophenyl)-3-hydroxy-1-methyl-1H-1,4-benzodiazepin-2(3H)-one; lormetazepam; 5-(4-chlorophenyl)-2,5-dihydro-3H-imidazo[2,1a]isoindol-5-ol; mazindol; 7-chloro-2,3-dihydro-1-methyl-5-phenyl-1H-1,4-benzodiazepine; medazepam; N-(3-chloropropyl)-α-methylphenethylamine; mefenorex; meperidine; 2-methyl-2-propyltrimethylene dicarbamate; meprobamate; meptazinol; metazocine; methylmorphine; N,α-dimethylphenethylamine; metamphetamine; (±)-6-dimethylamino-4,4-diphenol-3-heptanone; methadone; 2-methyl-3-o-tolyl-4(3H)-quinazolinone; methaqualone; methyl [2-phenyl-2-(2-piperidyl)acetate]; methylphenidate; 5-ethyl-1-methyl-5-phenylbarbituric acid; methylphenobarbital; 3,3-diethyl-5-methyl-2,4-piperidinedione; methyprylon; metopon; 8-chloro-6-(2-fluorophenyl)-1-methyl-4H-imidazo[1,5-a][1,4]benzodiazepine; midazolam; 2-(benzhydrylsulfinyl)acetamide; modafinil; (5α,6α)-7,8-didehydro-4,5-epoxy-17-methyl-7-methylmorphinan-3,6-diol; morphine; myrophine; (±)-trans-3-(1,1-dimethylheptyl)-7,8,10,10α-tetrahy dro-1-hydroxy-6,6-dimethyl-6H-dibenzo-[b, d]pyran-9(6αH)one; nabilone; nalbuphene; nalorphine; narceine; nicomorphine; 1-methyl-7-nitro-5-phenyl-1H-1,4-benzodiazepin-2(3H)-one; nimetazepam; 7-nitro-5-phenyl-1H-1,4-benzodiazepin-2(3H)-one; nitrazepam; 7-chloro-5-phenyl-1H-1,4-benzodiazepin-2(3H)-one; nordazepam; norlevorphanol; 6-dimethylamino-4,4-diphenyl-3-hexanone; normethadone; normorphine; norpipanone; opium; 7-chloro-3-hydroxy-5-phenyl-1H-1,4-benzodiazepin-2(3H)-one; oxazepam; (cis-/trans-)-10-chloro-2,3,7,11b-tetrahydro-2-methyl-11b-phenyloxazolo[3,2-d][1,4]benzodiazepin-6-(5H)-one; oxazolam; 4,5α-epoxy-14-hydroxy-3-methoxy-17-methyl-6-morphinanone; oxycodone; oxymorphone; papaveretum; 2-imino-5-phenyl-4-oxazolidinone; pemoline; 1,2,3,4,5,6-hexahydro-6,11-dimethyl-3-(3-methyl-2-butenyl)-2,6-methano-3-benzazocin-8-ol; pentazocine; 5-ethyl-5-(1-methylbutyl)-barbituric acid; pentobarbital; ethyl-(1-methyl-4-phenyl-4-piperidinecarboxylate); pethidine; phenadoxone; phenomorphane; phenazocine; phenoperidine; piminodine; pholcodeine; 3-methyl-2-phenylmorpholine; phenmetrazine; 5-ethyl-5-phenylbarbituric acid; phenobarbital; α,α-dimethylphenethylamine; phentermine; (R)-3-[-1-hydroxy-2-(methylamino)ethyl]phenol; phenylephrine, 7-chloro-5-phenyl-1-(2-propynyl)-1H-1,4-benzodiazepin-2(3H)-one; pinazepam; α-(2-piperidyl)benzhydryl alcohol; pipradrol; 1′-(3-cyano-3,3-diphenylpropyl)[1,4′-bipiperidine]-4′-carboxamide; piritramide; 7-chloro-1-(cyclopropylmethyl)-5-phenyl-1H-1,4-benzodiazepin-2(3H)-one; prazepam; profadol; proheptazine; promedol; properidine; propoxyphene; N-(1-methyl-2-piperidinoethyl)-N-(2-pyridyl)propionamide; methyl {3-[4-methoxycarbonyl-4-(N-phenylpropanamido)piperidino]propanoate}; (S,S)-2-methylamino-1-phenylpropan-1-ol; pseudoephedrine, remifentanil; 5-sec-butyl-5-ethylbarbituric acid; secbutabarbital; 5-allyl-5-(1-methylbutyl)-barbituric acid; secobarbital; N-{-4-methoxymethyl-1-[2-(2-thienyl)ethyl]-4-piperidyl}propionanilide; sufentanil; 7-chloro-2-hydroxymethyl-5-phenyl-1H-1,4-benzodiazepin-2(3H)-one; temazepam; 7-chloro-5-(1-cyclohexenyl)-1-methyl-1H-1,4-benzodiazepin-2(3H)-one; tetrazepam; ethyl (2-dimethylamino-1-phenyl-3-cyclohexene-1-carboxylate; cis-/trans-tilidine; tramadol; 8-chloro-6-(2-chlorophenyl)-1-methyl-4H-[1,2,4]triazolo[4,3-a][1,4]benzodiazepine; triazolam; 5-(1-methylbutyl)-5-vinylbarbituric acid; vinylbital; (1R*,2R*)-3-(3-dimethylamino-1-ethyl-2-methylpropyl)phenol; (1R,2R,4S)-2-(dimethylamino)methyl-4-(p-fluorobenzyloxy)-1-(m-methoxyphenyl)cyclohexanol.

In addition to the above compounds, active pharmaceutical ingredients also include a prodrug of any of these compounds. The term “prodrug” means a compound that is a metabolic precursor to the active pharmaceutical ingredient. This precursor is transformed in vivo to provide the active pharmaceutical ingredient which has the desired therapeutic effect.

The dosage forms according to the disclosure include various active agents and their pharmaceutically acceptable salts thereof. Pharmaceutically acceptable salts include, but are not limited to, inorganic acid salts such as hydrochloride, hydrobromide, sulfate, phosphate and the like; organic acid salts such as formate, acetate, trifluoroacetate, maleate, tartrate and the like; sulfonates such as methanesulfonate, benzenesulfonate, p-toluenesulfonate, and the like; amino acid salts such as arginate, asparginate, glutamate and the like, and metal salts such as sodium salt, potassium salt, cesium salt and the like; alkaline earth metals such as calcium salt, magnesium salt and the like; organic amine salts such as triethylamine salt, pyridine salt, picoline salt, ethanolamine salt, triethanolamine salt, dicyclohexylamine salt, N,N′-dibenzylethylenediamine salt and the like.

The phrase “pharmaceutically acceptable” means that which is useful in preparing a pharmaceutical composition that is generally safe, non-toxic, and is not biologically or otherwise undesirable and is acceptable for human pharmaceutical use.

Furthermore, in addition to the above compounds, active pharmaceutical ingredients also include solvates of any of the above-mentioned compounds. The term “solvate” refers to an aggregate that comprises one or more molecules of active pharmaceutical ingredient with one or more molecules of a solvent. The solvent may be water, in which case the solvate may be a hydrate. Alternatively, the solvent may be an organic solvent. In one embodiment, “solvate” refers to the active pharmaceutical ingredient in its state prior to dissolution. Alternatively, the solid particles of a suspended active pharmaceutical ingredient may comprise a co-precipitated solvent.

As used herein, the terms “therapeutically effective” and an “effective amount” refer to the amount of active agent or the rate at which it is administered which is needed to produce a desired therapeutic result.

As used herein, “shell” or “shell composition” refers to the shell or outer portion of a softgel capsule which encapsulates a fill composition.

The term “fill material” may be used interchangeably with the terms “fill composition,” and “fill” throughout the description. These terms refer to the inner portion of the softgel capsule that is encapsulated by the shell composition.

As used herein, “conventional pH dependent polymers” refer to, but are not limited to, acrylic and methacrylic acid polymers, which may be available under the tradename EUDRAGIT® and other conventional acid insoluble polymers, e.g., methyl acrylate-methacrylic acid copolymers. Other conventional acid insoluble polymers include, without limitation, cellulose acetate succinate, cellulose acetate phthalate, cellulose acetate butyrate, hydroxypropyl methyl cellulose phthalate, hydroxy propyl methyl cellulose acetate succinate (hypermellose acetate succinate), polyvinyl acetate phthalate (PVAP), algenic acid salts such as sodium alginate and potassium alginate, stearic acid, and shellac. Pectin and pectin derivatives are not considered to be conventional pH dependent polymers. Gellan gum and its derivatives are also not considered to be conventional pH dependent polymers. In some embodiments, the pH dependent shell composition of the present disclosure does not include an acid insoluble polymer. In other words, in certain embodiments, the pH dependent shell composition and the pH dependent softgel capsule are “free or substantially free of conventional pH dependent polymers.”

As used herein, “free or substantially free,” refers to a composition that comprises less than about 1 wt. %, less than about 0.5 wt. %, less than about 0.25 wt. %, less than about 0.1 wt. %, less than about 0.05 wt. %, less than about 0.01 wt. %, or 0 wt. % of said component.

All references to “molecular weight” herein refer to number average molecular weights unless otherwise specified.

The term “ambient temperature” as used herein refers to a temperature of about 20-35° C.

All references to wt. % throughout the specifications and the claims refer to the weight of the component in reference to the weight of the entire subject composition and may also be designated as w/w, unless explicitly indicated otherwise.

As used herein, “delayed release capsules” or “delayed release softgel capsules” or “pH dependent capsules” or “pH dependent softgel capsules” refer to capsules which have delayed or pH dependent properties once the fill composition is encapsulated in the pH dependent shell composition, and the capsules are dried. In certain embodiments, these terms may refer to capsules that have also been cured after drying. In certain embodiments, no further processing steps past drying are required. In certain embodiments, no further processing steps past curing are required. The term “cure” and variations thereof may be used interchangeably with the term “anneal” and variations thereof.

As used herein, “modified release softgel capsules” refer to capsules which have a controlled release fill composition encapsulated in a pH dependent shell composition.

As used herein, the term “controlled release,” refers to an active agent that is released over a period of time, e.g., to provide a once daily or twice daily dosage form.

As used herein, “about” refers to any values that are within a variation of ±10%, such that “about 10” would include from 9 to 11. As used herein, “a,” “an,” or “the” refers to one or more, unless otherwise specified. Thus, for example, reference to “an excipient” includes a single excipient as well as a mixture of two or more different excipients, and the like.

Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context.

The use of any and all examples, or exemplary language (e.g., “such as”) provided herein, is intended merely to illuminate certain materials and methods and does not pose a limitation on scope. No language in the specification should be construed as indicating any non-claimed element as essential to the practice of the disclosed materials and methods.

### Controlled Release Fill Composition

According to embodiments, the fill composition of the modified release softgel capsules described herein is a controlled release fill composition that includes at least one active agent and a controlled release material.

The active agent may be any of the active agents described hereinbefore, such as, without limitations, a pharmaceutically active ingredient or a nutraceutical (e.g., vitamins, minerals, or supplements). Particularly suitable active agents are those that benefit from a controlled release over an extended duration, e.g., 12 hours or 24 hours, for a once daily or a twice daily administration. Active agents that are unstable or could benefit from protection from the low pH in the gastric fluid in the stomach area (such as peptides, proteins, enzymes, and the like) could also be advantageously utilized with the softgel capsules described herein. Additionally, active agents that may irritate or damage the gastric mucosa (e.g., NSAIDs) may be incorporated in the softgel capsules described herein without the need for costly tableting and coating processes. In general, any active agent that could benefit from an ability of the softgel capsule to facilitate targeted release of the active agent to a specific area within the gastrointestinal tract (GIT) and from an ability of the softgel capsule to control the release of the active agent at the specific area within the GIT, are encompassed herein.

In one embodiment, the active agent includes fish oil, garlic oil, krill oil, or any other active agent that could create an unpleasant sensation due to premature release, e.g., belching, of the active agent.

In one embodiment, the active agent includes NSAIDs, such as, ibuprofen, or any other active agent that could irritate the gastric mucosa due to premature release of the active agent in the stomach area rather than at a later point in the GIT.

In one embodiment, the active agent includes peptides, proteins, enzymes, or any other active agent that may be unstable in the acidic environment of the stomach and/or may be better absorbed closer to the colon area.

In certain embodiments, the active agent includes anti-histamines (such as diphenhydramine) or acetaminophen.

In one embodiment, the active agent includes drugs susceptible to abuse. In an alternative embodiment, the active agent is a drug that is not susceptible to abuse.

To the extent that some examples are shown with respect to certain active agents, these examples should not be construed as limited to only those active agents, but may be viewed as a proof of concept that may be applicable to a variety of active agents.

In certain embodiments, the active agent is present in the controlled release fill composition in an amount of at least about 1 wt. %, at least about 5 wt. %, at least about 10 wt. %, at least about 15 wt. %, at least about 20 wt. %, at least about 25 wt. %, or at least about 30 wt. % and up to about 35 wt. %, up to about 40 wt. %, up to about 45 wt. %, up to about 50 wt. %, up to about 55 wt. %, or up to about 60 wt. %, based on a total weight of the controlled release fill composition. In certain embodiments, the controlled release fill composition includes from about 12 wt. % to about 18 wt. %, from about 19 wt. % to about 25 wt. %, from about 24 wt. % to about 32 wt. %, from about 4 wt. % to about 10 wt. %, or from about 25 wt. % to about 42 wt. % active agent, based on total weight of the controlled release fill composition. In one embodiment, the active agent is present in the controlled release fill composition in an amount of from about 5 wt. % to about 60 wt. %, based on the total weight of the controlled release fill composition. In one embodiment, the active agent is present in the controlled release fill composition in an amount of from about 10 wt. % to about 30 wt. %, based on the total weight of the controlled release fill composition. The concentration ranges of the active agent described herein may refer to a concentration of a single active agent (regardless of the number of active agents in the fill composition) or to the cumulative concentration of all active agents in the fill composition (if more than one active agent is present in the fill composition).

In certain embodiments, controlled release materials that may be incorporated into the controlled release fill composition include, without limitations, polyethylene oxide, cellulose derivatives, a gum, or a combination thereof.

In embodiments, polyethylene oxides that may be utilized have a number average molecular weight that ranges from any one of about 0.05M, about 0.5M Dalton, about 1M Dalton, about 2M Dalton, about 3M Dalton, or about 4M Dalton to any of about 5M about, 7M Dalton, about 10M Dalton, about 12M Dalton, about 15M Dalton, or about 20M Dalton, or any sub-range or single value therein. In one embodiment, the number average molecular weight of the polyethylene oxide in the controlled release fill composition ranges from about 0.05M Dalton to about 15M Dalton. In one embodiment, the number average molecular weight of the polyethylene oxide in the controlled release fill composition ranges from about 1M Dalton to about 10M Dalton. In one embodiment, the number average molecular weight of the polyethylene oxide in the controlled release fill composition ranges from about 2M Dalton to about 5M Dalton.

Suitable polyethylene oxides are typically non-ionic, high molecular weight, water-soluble polyethylene oxide resins. Exemplary PEO resins of this type are the Polyox™ water-soluble resins available from DuPont Pharma Solutions. These PEO resins are typically used as thickeners and rheology control agents. In the present disclosure, these water-soluble PEO resins can be employed to modify or control the release of the active agent from the fill composition. PEO resins may also be employed in the fill compositions to deter abuse of the API that is contained in the fill compositions if the API is susceptible to abuse.

A significant advantage of the use of polyethylene oxide as a rate controlling component of the fill composition is that it does not tend to be as tacky or sticky as other rate-controlling polymers thereby facilitating the encapsulation process and ensuring a more homogeneous fill composition. While other additional rate-controlling polymers can be employed, the amounts of such rate-controlling polymers must be carefully selected to prevent this stickiness or tackiness from causing problems during the encapsulation process that may lead to an inferior product.

In embodiments, cellulose derivatives that may be utilized include microcrystalline cellulose, sodium carboxymethyl cellulose, methylcellulose, ethyl cellulose, hydroxyethyl cellulose, hydroxypropyl cellulose, hydroxypropyl methylcellulose, or combinations thereof. In one embodiment, the cellulose derivative is hydroxypropyl methylcellulose.

In embodiments, gums that may be utilized include gum tragacanth, gum acacia, agar, tara gum, karaya, gellan gum, welan gum, rhamsan gum, guar gum, xanthan gum, locust bean gum, or combinations thereof. In one embodiment, the gum includes xanthan gum, locust bean gum, or combinations thereof. In one embodiment, the gum includes xanthan gum. In one embodiment, the gum includes locust bean gum.

Optional additional release controlling polymers may include pectin, starch, carbomer, sodium alginate, gelatin, casein, carrageenans, collagen, dextran, succinoglucon, polyvinyl alcohol clays, and combinations thereof.

In certain embodiment, the controlled release material includes any of the polyethylene oxides described herein by itself. In certain embodiments, the controlled release material includes any of the polyethylene oxides described herein in combination with any of the cellulose derivatives described herein. In certain embodiments, the controlled release material includes any of the polyethylene oxides described herein in combination with any of the gum described herein.

In embodiments, the controlled release material is in the controlled release fill composition in an amount of at least about 8 wt. %, at least about 10 wt. %, at least about 12 wt. %, at least about 14 wt. %, at least about 16 wt. %, at least about 18 wt. %, or at least about 20 wt. % up to about 25 wt. %, up to about 35 wt. %, up to about 45 wt. %, up to about 55 wt. %, or up to about 65 wt. %, or any sub-range therein, based on a total weight of the controlled release fill composition. In certain embodiments, the controlled release fill composition includes from about 8 wt. % to about 15 wt. %, from about 16 wt. % to about 20 wt. %, from about 22 wt. % to about 28 wt. %, from about 15 wt. % to about 30 wt. %, from about 20 wt. % to about 42 wt. %, from about 10 wt. % to about 35 wt. %, or from about 11 wt. % to about 40.5 wt. % controlled release material, based on total weight of the controlled release fill composition.

In one embodiment, the controlled release fill material (e.g., PEO) is employed in the controlled release fill composition in an amount of at least 21.5 wt. %, based on the total weight of the controlled release fill composition. In one embodiment, the controlled release fill material (e.g., PEO) is in present in the controlled release fill composition in an amount from about 10 wt. % to about 65 wt. %, based on the total weight of the controlled release fill composition. In one embodiment, the controlled release fill material (e.g., PEO) is present in the controlled release fill composition in an amount of about 25 wt. % to about 40 wt. %, based on the total weight of the controlled release fill composition.

In an alternative embodiment, PEO may be present in the controlled release fill composition in any suitable amount when the hydrophilic carrier is present in an amount of up to 65 wt. %, based on the total weight of the controlled release fill composition. In this embodiment, the minimum amount of hydrophilic carrier may optionally be at least about 30 wt. %, or at least about 40 wt. %, or at least about 55 wt. %, based on the total weight of the controlled release fill composition. In these alternative embodiments, the amount of PEO in the controlled release fill composition can be from about 5 wt. % to about 35 wt. %, or about 20 wt. %, based on total weight of the controlled release fill composition.

The concentration ranges of the controlled release material described herein may refer to a concentration of a single controlled release material (regardless of the number of controlled release materials in the fill composition) or to the cumulative concentration of all controlled release materials in the fill composition (if more than one controlled release material is present in the fill composition).

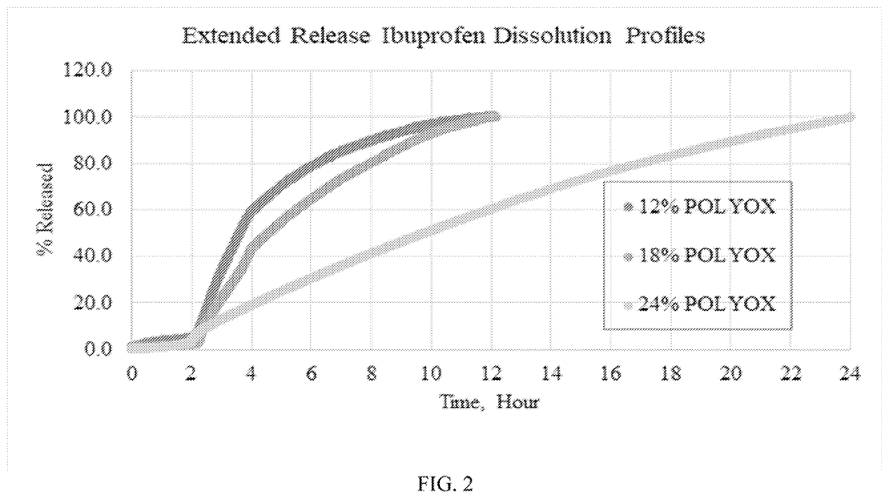

The concentration of the controlled release material in the fill composition may be modified to attain a target release profile for the active agent. For instance, as illustrated in example 11 and in FIG. 2, the amount of polyethylene oxide, in certain embodiments, effects the release rate of the active agent in the controlled release fill composition. A polyethylene oxide, having a number average molecular weight of 4M Dalton, achieved a 12 hour release profile of an active agent when incorporated into the fill composition at a concentration of about 12 wt. % and about 18 wt. %, based on total weight of the fill composition. In comparison, the same polyethylene oxide achieved a 24 hour zero order release profile of an active agent when incorporated into the fill composition at a concentration of about 24 wt. %, based on total weight of the fill composition.

Similarly, the weight ratio of the controlled release material to other components of the fill composition (such as the active agent or the hydrophilic carrier if present) may be adjusted to attain a target release profile for the active agent. In certain embodiments, the wt:wt ratio of the controlled release to the active agent may range from about 10:1 to about 1:10, about 8:1 to about 1:8, about 5:1 to about 1:5, about 3:1 to about 1:3, or about 1:1.

In certain embodiments, the controlled release material (e.g., PEO) and the water and/or hydrophilic carrier may be present in the controlled release fill composition in any suitable amount such that the weight ratio of the controlled release material (e.g., PEO) to the water and/or the hydrophilic carrier (individually or cumulatively) ranges from about 10:1 to about 1:10, from about 8:1 to about 1:8, from about 5:1 to about 1:5, from about 3:1 to about 1:3, from about 2:1 to about 1:2, from about 10:1 up to 1:3, from about 8:1 up to 1:3, from about 5:1 up to 1:3, from about 3:1 up to 1:3, from about 2:1 up to 1:3, from about 1:1 up to 1:3, from about 10:1 to about 1:2, from about 8:1 to about 1:2, from about 5:1 to about 1:2, from about 3:1 to about 1:2, from about 1:1 to about 1:2, or any sub-range or single weight ratio value therein. In one embodiment, the weight ratio of the controlled release material (e.g., PEO) to the water and/or the hydrophilic carrier (individually or cumulatively) ranges from about 2:1 to about 1:2. In one embodiment, the weight ratio of the controlled release material (e.g., PEO) to the water and/or the hydrophilic carrier (individually or cumulatively) ranges from about 3:1 up to 1:3.

Other factors, such as, without limitations, the type of controlled release material and the molecular weight of the controlled release material, may also affect the release rate of an active agent in the fill composition.

In certain embodiment, the controlled release fill composition may further include a hydrophilic carrier. The hydrophilic carrier maybe a low molecular weight polyol, such as, polyethylene glycol, polypropylene glycol, or a combination thereof. The hydrophilic carrier may also be water. Examples of additional suitable hydrophilic carriers are hydrophilic solvents that include polyoxyethylene derivatives of a sorbitan ester, such as sorbitan monolaurate (Polysorbate 20), Polysorbate 80, Polysorbate 60, polyoxyethylene 20 sorbitan trioleate (Polysorbate 85), acetic acid, formic acid, other hydrophilic surfactants and mixtures thereof.

In certain embodiments, the controlled release fill composition includes a hydrophilic carrier having a number average molecular weight of from any of about 200 Dalton, about 400 Dalton, about 600 Dalton, about 800 Dalton, or about 1000 Dalton to any of about 2000 Dalton, about 3000 Dalton, about 4000 Dalton, about 5000 Dalton, about 6000 Da, or about 7000 Da, or any sub-range or single value therein. Exemplary hydrophilic carriers that may be utilized in the controlled release fill composition include polyethylene glycol 400, polyethylene glycol 600, water, or a combination thereof.

In certain embodiments, the hydrophilic carrier in the fill composition comprises polyethylene glycol having a number average molecular weight of from 300 Dalton to about 7000 Dalton. In certain embodiments, the hydrophilic carrier in the fill composition has a number average molecule weight of from about 200 daltons to 5000 daltons, more preferably, the number average molecular weight of the hydrophilic carrier is from about 300 daltons to about 3000 daltons, and most preferably the number average molecule weight of the hydrophilic carrier is from about 400 daltons to about 1500 daltons. In certain embodiments, the hydrophilic carrier may include compounds with a number average molecular weight that is below 200 daltons.

In certain embodiments, the hydrophilic carrier is present in the controlled release fill composition in an amount of above 0 wt. %, at least about 15 wt. %, or at least about 30 wt. % up to about 45 wt. %, up to about 60 wt. %, up to about 70 wt. %, or up to about 80 wt. %, based on a total weight of the controlled release fill composition. In certain embodiments, the controlled release fill composition includes from about 5 wt. % to about 15 wt. %, from about 15 wt. % to about 28 wt. %, from about 20 wt. % to about 32 wt. %, from about 20 wt. % to about 42 wt. %, from about 22 wt. % to about 45 wt. %, from about 40 wt. % to about 45 wt. %, from about 40 wt. % to about 55 wt. %, from about 35 wt. % to about 55 wt. %, from about 56 wt. % to about 77 wt. %, from about 40 wt. % to about 79 wt. %, or from about 29 wt. % to about 66 wt. % hydrophilic carrier, based on total weight of the controlled release fill composition.

In one embodiment, the hydrophilic carrier is included in the controlled release fill composition in an amount of up to 65 wt. %, based on the total weight of the controlled release fill composition. In another embodiment, the hydrophilic carrier is included in the controlled release fill composition in an amount of from about 10 wt. % to about 75 wt. %, or 30 wt. % to about 70 wt. %, based on the total weight of the controlled release fill composition. Preferably, the hydrophilic carrier is included in the controlled release fill composition in an amount of from about 40 wt. % to about 60 wt. %, based on the total weight of the controlled release fill composition.

In another embodiment, the hydrophilic carrier can be present in the controlled release fill composition in any amount so long at the controlled release material (e.g., polyethylene oxide) is present in an amount of at least 21.5 wt. %, based on the total weight of the controlled release fill composition. In this embodiment, the hydrophilic carrier is typically present in amounts of up to 65 wt. %, or from 10 wt. % to 65 wt. %, or from 30 wt. % to 60 wt. %, or from 30 wt. % to 55 wt. %, based on the total weight of the controlled release fill composition. The hydrophilic carrier is used to dissolve, disperse and/or suspend the other components of the liquid fill composition in a liquid and may also function to adjust the viscosity of the liquid fill composition to a desired viscosity for the encapsulation step.

The concentration ranges of the hydrophilic carrier described herein may refer to a concentration of a single hydrophilic carrier material (regardless of the number of hydrophilic carrier materials in the fill composition) or to the cumulative concentration of all hydrophilic carrier materials in the fill composition (if more than one hydrophilic carrier material is present in the fill composition).

In certain embodiments, the fill composition is a liquid with a viscosity in the range of 1000 cP to 100,000 cP, or from 5,000 cP to 80,000 cP, or from 10,000 cP to 60,000 cP at the time of filling (or encapsulation within) the capsule shell composition. The viscosity of the liquid fill composition was determined at 20° C. using a HAAKE RheoStress 600 rheometer equipped with a 40 mm flat plate geometry. The geometry oscillated at 1 Hz with a gap setting of 2 mm. A significant advantage of the fill composition being liquid during processing, is that it obviates the need to handle powders in the process for making the dosage form, except in the initial mixing step, in contrast to tablet dosage forms which generally require handling of powders throughout the process of making the dosage form. Further, processing of the liquid fill compositions described herein gets away from the need to include flowability enhancer or processability enhancers to facilitate processing. Similarly, given that the fill compositions may be liquid at ambient temperatures, there is no need to heat them prior to encapsulation, which could be harmful to heat sensitive materials such as those utilized in shell compositions of certain softgel capsules.

In certain embodiments, the controlled release fill composition releases less than about 85%, less than about 80%, less than about 75%, less than about 70%, less than about 65%, less than about 60%, less than about 55%, less than about 50%, less than about 45%, less than about 40%, less than about 35%, or less than about 30% of the active agent after about 0.5 hour, about 1 hour, about 2 hours, about 3 hours, about 4 hours, or about 5 hours in pH 6.8 phosphate buffer, optionally with pancreatin, based on a fiber optic dissolution test using a USP Apparatus II with a paddle speed of 50 rpm.

In certain embodiments, the controlled release fill composition releases about 10 wt. % to about 30 wt. % of active agent at 1 hours, about 15 wt. % to about 50 wt. % of active agent at 2 hours, about 20 wt. % to about 80 wt. % of active agent at 4 hours, about 40 wt. % to about 95 wt. % of active agent at 8 hours, from about 65 wt. % to about 100 wt. % at 12 hours, and greater than 90 wt. % of active agent at 24 hours, in each case, as measured by an in-vitro dissolution in a fiber optic dissolution test using USP Apparatus II (paddle) at 50 rpm, in biological, artificial, or simulated gastric fluid, such as 0.1 N HCl and/or biological, artificial, or simulated intestinal fluid, such as pH 6.8 phosphate buffer and/or water, optionally with pancreatin, at 37° C.

In certain embodiments, the active agent release rate from the controlled release fill composition is such that less than 80%, less than about 75%, less than about 70%, less than about 65%, less than about 60%, less than about 55%, less than about 50%, less than about 45%, less than about 40%, less than about 35%, or less than about 30% of the active agent is released after about 0.5 hour, about 1 hour, about 2 hours, about 3 hours, about 4 hours, or about 5 hours in a fiberoptic dissolution test using USP Apparatus II using a paddle speed of 100 RPM at 37° C. in 500 ml of biological, artificial, or simulated gastric fluid, such as 0.1 N HCl and/or biological, artificial, or simulated intestinal fluid, such as pH 6.8 phosphate buffer and/or water.

As will be described in further detail below, the modified release softgel capsule may be annealed at an annealing temperature for an annealing duration. In certain embodiments, the annealing facilitates formation of a matrix inside the modified release softgel capsule. For instance, when the controlled release material is polyethylene oxide, the annealing may facilitate melting of the polyethylene oxide to form a matrix (liquid or solid) that is encapsulated inside the pH dependent shell composition.

Another embodiment relates to a method of producing a controlled release fill composition containing a polyethylene oxide resin. This process is designed to accommodate softgel capsule shell compositions which are not compatible with high encapsulation temperatures due to the relatively low melting points of the capsule shell composition. For example, gelatin-based softgels may begin to melt at temperatures of from 33-45° C., depending to some extent on the water content of the capsule shell material at the time of encapsulation. For such lower melting temperature capsule shell materials, a method has been devised to fill the capsules with a liquid fill composition at lower temperatures. A significant advantage of this method is that it can be used to ultimately encapsulate a highly viscous liquid or semi-solid or solid fill composition. In this method, a solid solution or semi-solid fill is formed in situ inside the capsule as a result of the heating step carried out after encapsulation.

In this method, suspensions and dispersions, rather than a solution, can be employed. A softgel capsule shell will typically contain water in amounts of up to 20 wt. %, based on the total weight of the capsule shell, upon completion of the encapsulation step. During the encapsulation and subsequent drying step, a significant portion, i.e. up to about 70%, of the water in the capsule shell will migrate into the fill composition and solubilize solid components within the suspension/dispersion of the fill composition, like PEO, in situ to form the desired solution. With this method, solubilization of solid components within the fill composition (e.g., PEO) occurs in situ. The water content in the fill composition, prior to encapsulation, is sufficiently low to limit or avoid solubilization of at least some of the constituents of the fill composition (such as PEO) prior to the encapsulation and drying steps. Premature solubilization of certain constituents within the fill composition (i.e., before encapsulation and drying), could increase the viscosity of the fill composition and hinder processability. Typically, the initial fill composition will have a water content of about 2 wt. % to about 10 wt. %, based on total weight of the fill composition, to avoid premature solubilization of the PEO component of the fill composition prior to encapsulation. After encapsulation of the fill composition, a portion of the water from the softgel capsule shell migrates into the fill composition, typically raising the water content of the fill composition to from about 15 wt. % to about 20 wt. %, based on total weight of the encapsulated fill composition, thereby causing solubilization of the PEO in the encapsulated fill composition. During the subsequent drying water is gradually removed until the water content of the encapsulated fill composition falls below 10 wt. %, based on total weight of the encapsulated and dried fill composition. After the final heating step (also referred to as annealing step), the water content of the final encapsulated fill composition is further reduced to from about 5 wt. % to about 8 wt. %, based on total weight of the final encapsulated fill composition. The final encapsulated fill composition forms a solid solution of PEO in the hydrophilic carrier.

This process of forming the solid solution in situ is important since it provides a more uniform distribution of the API in the fill composition, unlike powder filled capsules or other solid dosage forms. Uniform distribution of the API is an important characteristic for delivery of high potency and/or low dose API's since such API's should be delivered at a relatively constant rate over time to avoid over or under dosing. In certain embodiments, the uniform distribution of the API in the fill composition enables zero order release of the API from the controlled release fill composition (where the API is delivered at a relatively constant rate over time, e.g., from about 2 hours to about 12 hours, or from about 2 hours to about 24 hours).

In an embodiment, the controlled release fill composition comprises, consists, or consists essentially of at least one active agent (e.g., pharmaceutically active ingredient such as NSAIDs (e.g., ibuprofen), anti-histamines (e.g., diphenhydramine), acetaminophen, nutraceuticals (e.g., garlic oil, fish oil, krill oil, or other vitamins, minerals, or supplements)), a controlled release material (e.g., polyethylene oxide with a number average molecular weight of about 0.05M Dalton to about 15M Dalton optionally in combination with a cellulose derivative (e.g., hydroxypropyl methylcellulose) or a gum (e.g., xanthan gum)), and a hydrophilic carrier (e.g., polyethylene glycol with a number average molecular weight of from about 200 Dalton to about 5000 Dalton optionally in combination with water).

In certain embodiments, the controlled release fill compositions may include additional fill components such as flavoring agents, sweetening agents, coloring agents and fillers or other pharmaceutically acceptable excipients or additives such as synthetic dyes and mineral oxides.

### pH Dependent Shell Composition

According to an embodiment, the pH dependent shell composition comprises gelatin, dextrose, a pH dependent material (e.g., a low methoxyl pectin) and optionally a plasticizer. Preferably, the pH dependent shell composition is free of additional pH dependent polymers.

In an embodiment, the gelatin in the pH dependent shell composition may include Type A gelatin, Type B gelatin, a hide or skin gelatin (e.g., bovine skin, pig skin) and/or a bone gelatin (e.g., bovine bone, pig bone) used alone or in combination. In one embodiment, the gelatin is a 250 Bloom gelatin. In one embodiment, the gelatin is a 150 Bloom gelatin. In another embodiment, there is only one type of gelatin. In yet another embodiment, the gelatin is a combination of at least two types of gelatins. In an embodiment, the amount of gelatin in the pH dependent shell composition is from about 25 wt. % to about 85 wt. %, from about 25 wt. % to about 80 wt. % from about 30 wt. % to about 85 wt. %, from about 30 wt. % to about 75 wt. %, from about 35 wt. % to about 70 wt. %, from about 30 wt. % to about 65 wt. %, from about 40 wt. % to about 65 wt. %, from about 30 wt. % to about 55 wt. %, from about 30 wt. % to about 40 wt. %, about 40 wt. % to about 80 wt. %, from about 45 wt. % to about 65 wt. %, from about 45 wt. % to about 60 wt. %, from about 45 wt. % to about 75 wt. %, or from about 50 wt. % to about 70 wt. %, or any single value or sub-range therein, based on total weight of the dry capsule shell composition.

In one embodiment, the pH dependent capsule shell composition comprises dextrose. In an embodiment, the amount of dextrose in the pH dependent capsule shell composition is from about 0.001 wt. % to about 1.0 wt. %, from about 0.002 wt. % to about 0.008 wt. %, from about 0.005 wt. % or about 0.01 wt. % to about 4 wt. %, from about 0.1 wt. % or about 0.15 wt. % to about 3 wt. %, from about 0.1 wt. % to about 1 wt. %, from about 0.1 or about 0.15 wt. % or about 0.2 wt. % or about 0.25 wt. % to about 2 wt. %, from about 0.1 wt. % to about 0.2 wt. %, from about 0.1 wt. % to about 0.4 wt. %, from about 0.05 wt. % to about 0.5 wt. %, or any single value or sub-range therein, based on total weight of the dry capsule shell composition. The dextrose may be added to the pH dependent shell composition to mitigate potential reduction in gel strength. Without being construed as limiting, it is believed that the dextrose interacts with the gelatin in the shell composition and causes the gelatin to cross-link. The effect of the amount of dextrose on the dissolution properties of the shell composition is further illustrated in the examples. The concentration of dextrose in the pH dependent shell composition may be in an effective amount to improve the gel strength but not so high that it would interfere with the seal of the capsule or manufacturability or the product performance.

In some embodiments, the pH dependent shell composition may comprise pectin, e.g., a low methoxyl pectin. In an embodiment, the pectin is low methylester (LM) pectin with Degree of Esterification lower than 50. In some embodiments, the pectin is amidated pectin. In other embodiments, the low methoxyl (LM) pectin is non-amidated pectin. In certain embodiments, the pectin is a combination of amidated pectin and non-amidated pectin. The addition of pectin contributes to the pH dependent nature of the shell composition.

Too much pectin in the dosage form may reduce the gel strength of the shell composition which may in turn adversely affect the sealability of the softgel capsule. Too much pectin in the pH dependent shell composition may also increase the viscosity of the shell composition, making it challenging or impossible to process from a manufacturing standpoint.

Therefore, pectin may be added to the dosage form at a concentration that is sufficiently high to form a modified release softgel capsule and at the same time is sufficiently low to mitigate the reduction in gel strength and to mitigate viscosity increase to a level that would hinder manufacturability.

In an embodiment, an amount of pectin in the pH dependent shell composition is about 2 wt. % to about 20 wt. %, from about 3 wt. % to about 15 wt. %, from about 3 wt. % to about 18 wt. %, from about 5 wt. % to about 15 wt. %, from about 3 wt. % to about 5.5 wt. %, from about 3.5 wt. % to about 6.5 wt. %, from about 2.5 wt. % to about 7 wt. %, from about 4 wt. % to about 11 wt. %, from about 7 wt. % to about 12 wt. %, from about 8 wt. % to about 13 wt. %, or from about 5 wt. % to about 10 wt. %, or any single value or sub-range therein, based on total weight of the dry capsule shell composition.

The degree of esterification of the pectin incorporated in the pH dependent shell composition may be lower than about 50%, or may range from about 10% to about 50%, from about 20% to about 40%, or from about 25% to about 35%. Also, the pectin may be amidated or non-amidated.

In certain embodiments, the pH dependent shell composition comprises a stabilizer and/or a binder comprising gellan gum. In certain embodiments, the wt:wt ratio of pectin to stabilizer and/or binder (e.g., gellan gum) is about 1:10 to about 70:1; about 1:10 to about 50:1; about 1:5 to about 40:1; about 1:1 to about 25:1; about 1:1 to about 5:1; or about 10:1 to about 24:1.

In certain embodiments, the amount of stabilizer and/or binder (e.g., gellan gum) in the pH dependent shell composition is about 0.05 wt. % to about 5 wt. %, about 0.1 wt. % to about 3 wt. %, about 0.1 wt. % to about 2 wt. %, or about 0.2 wt. % to about 2 wt. % of stabilizer and/or binder (e.g., gellan gum), or any single value or sub-range therein, based on total weight of the dry capsule shell composition.

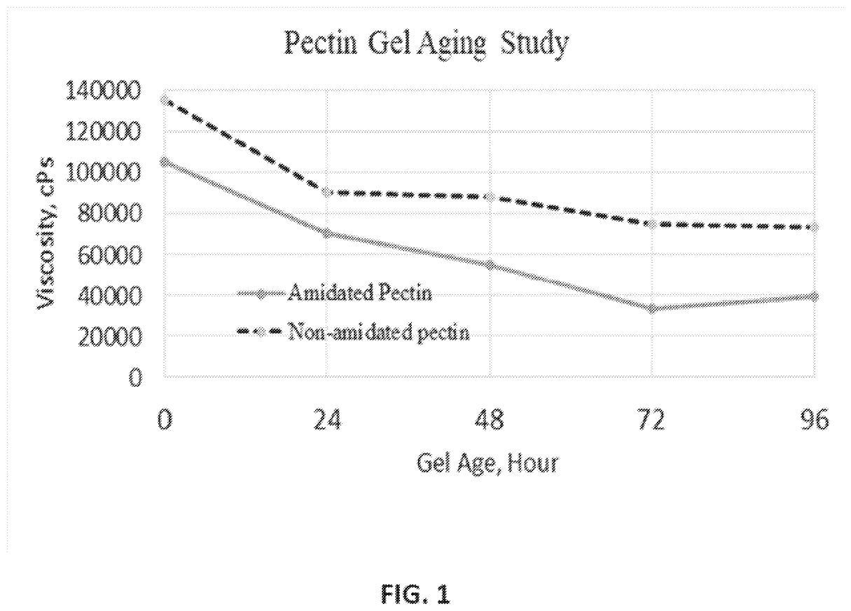

In certain embodiments, the pH dependent shell composition may have a viscosity ranging from any of about 20,000 cPs, about 30,000 cPs, about 40,000 cPs, about 50,000 cPs, about 60,000 cPs, or about 70,000 cPs to any of about 80,000 cPs, about 90,000 cPs, about 100,000 cPs, about 110,000 cPs, about 120,000 cPs, about 130,000 cPs, about 140,000 cPs, about 150,000 cPs, about 160,000 cPs, or about 170,000 cPs or any sub-range or single value therein. In one embodiment, the pH dependent shell composition has a viscosity ranging from about 100,000 cPs to about 130,000 cPs, or from about 110,000 cPs to about 125,000 cPs, or about 115,000 cPs, or about 120,000 cPs. The viscosity is measured using a rheometer at 60° C. as described in further detail in the examples related to FIG. 1. A gel mass sample (e.g., of any of the pH dependent shell compositions described herein) is loaded onto the platform of the rheometer, maintained at 60° C. A disc rotates at a certain speed to provide a fixed shear rate. The viscosity is obtained by measuring the shear stress and shear rate.

In certain embodiments, the pH dependent shell composition may maintain a viscosity that is suitable for manufacturability even after being aged in heat for up to about 24 hours, up to about 48 hours, up to about 72 hours, up to about 96 hours, or up to about 1 week. In certain embodiments, the viscosity of the pH dependent shell composition, after aging in heat (for up to about 24 hours, up to about 48 hours, up to about 72 hours, up to about 96 hours, or up to about 1 week) may reduce (from the viscosity value of the composition prior to aging) by up to about 80%, up to about 70%, up to about 60%, up to about 50%, up to about 40%, up to about 35%, or up to about 30%.

In an embodiment, the plasticizer in the pH dependent shell composition may include glycerol, sorbitol and combinations thereof. Other suitable plasticizers may include, but not be limited to, sugar alcohol plasticizer such as triacetin, isomalt, maltitol, xylitol, erythritol, adonitol, dulcitol, pentaerythritol, or mannitol; or polyol plasticizer such as diglycerin, ethylene glycol, diethylene glycol, triethyleneglycol, tetraethylene glycol, dipropylene glycol, a polyethylene glycol up to 10,000 MW, neopentyl glycol, propylene glycol, 1,3-propanediol, 2-methyl-1,3-propanediol, trimethylolpropane, a polyether polyol, ethanol amines; and mixtures thereof. Other exemplary plasticizers may also include, without limitations, low molecular weight polymers, oligomers, copolymers, oils, small organic molecules, low molecular weight polyols having aliphatic hydroxyls, ester-type plasticizers, glycol ethers, poly(propylene glycol), multi-block polymers, single block polymers, citrate ester-type plasticizers, and triacetin. Such plasticizers may include 1,2-butylene glycol, 2,3-butylene glycol, styrene glycol, monopropylene glycol monoisopropyl ether, propylene glycol monoethyl ether, ethylene glycol monoethyl ether, diethylene glycol monoethyl ether, sorbitol lactate, ethyl lactate, butyl lactate, ethyl glycolate, dibutyl sebacate, acetyltributylcitrate, triethyl citrate, glyceryl monostearate, polysorbate 80, acetyl triethyl citrate, tributyl citrate and allyl glycolate, and mixtures thereof.

In an embodiment, the amount of plasticizer in the pH dependent shell composition is from about 10 wt. % to about 40 wt. %, from about 15 wt. % to about 35 wt. %, from about 15 wt. % to about 45 wt. %, from about 15 wt. % to about 40 wt. %, from about 18 wt. % to about 45 wt. %, from about 18 wt. % to about 42 wt. %, from about 20 wt. % to about 35 wt. %, from about 20 wt. % to about 30 wt. %, from about 25 wt. % to about 30 wt. %, or any single value, or sub-range therein, based on total weight of the dry capsule shell composition.

In certain embodiments, the amount of the various components (e.g., pectin, dextrose, gelatin, plasticizer) and the ratio of the various components are tuned to control the dissolution and/or disintegration properties of the pH dependent shell composition across various pH ranges and correspondingly to facilitate targeted release of the active agent in specific area within the gastrointestinal tract.

For instance, the gelatin to pectin w:w ratio in the pH dependent shell composition may range from any of about 2:1, about 3:1, about 4:1, about 5:1, about 6:1, about 7:1, about 8:1, or about 9:1 to any of about 10:1, about 11:1, about 12:1, about 13:1, about 14:1, about 15:1, about 16:1, about 17:1, about 18:1, about 19:1, or about 20:1, or any sub-range or single value therein. In certain embodiments, lower gelatin to pectin w:w ratios provide for a pH dependent shell composition that is more stable (dissolves slower if at all) in acidic medium (e.g., 0.1N HCl optionally with Pepsin), while higher gelatin to pectin w:w ratios provide for a pH dependent shell composition that is less stable (dissolves faster) in acidic medium (e.g., 0.1N HCl optionally with Pepsin). The gelatin to pectin w:w ratio may be tuned to attain a particular dissolution time for softgel capsule in acidic medium (e.g., about 10 minutes, about 15 minutes, about 30 minutes, about 45 minutes, about 60 minutes, about 90 minutes, and so on).

The gelatin to plasticizer w:w ratio in the pH dependent shell composition may also be tuned to attain a particular capsule hardness level and may range from about 5:1 to about 1:5, from about 5:1 to about 1:2, from about 4:1 to about 1:4, from about 4:1 to about 1:2, from about 3:1 to about 1:3, from about 3:1 to about 1:2, from about 2:1 to about 1:2, about 1:1, or any single ratio value or sub-range therein.

In certain embodiments, the pH dependent shell compositions described herein may have a hardness ranging from any of about 5 N, about 6 N, about 7 N, about 8 N, about 9 N, or about 10 N to any of about 11 N, about 12 N, about 13 N, about 14 N, or about 15 N. The capsule hardness is determined using a hardness tester. The force required to cause a 2.0 mm deformation of the capsule in Newton is defined as the capsule hardness.

In certain embodiments, the pH dependent shell compositions described herein may have a shell moisture ranging from any of about 5%, about 6%, about 7%, about 8%, about 9%, or about 10% to any of about 11%, about 12%, about 13%, about 14%, or about 15%. The shell moisture is determined by loss on drying method. A pH dependent capsule shell composition sample of 1 to 2 grams is placed into a 105° C. oven for 17 hours. The initial weight of the sample is recorded. After drying the sample in the oven at 105° C. for 17 hours, the final weight of the sample is recorded. The percentage of weight loss, calculated in accordance with the below equation, is defined as the shell moisture:

$$\% weight lost = \frac{( initial weight ) - ( final weight )}{( initial weight )} \cdot 100 \%$$

In certain embodiments, the pH dependent shell compositions described herein may have an equilibrium relative humidity ranging from any of about 25%, about 28%, about 30%, about 32%, about 34%, or about 35% to any of about 38%, about 40%, about 42%, about 45%, or about 50%. Equilibrium Relative Humidity (%) is defined as the humidity condition at which the capsule maintained a constant total weight. It is determined using environmental chambers maintained at constant humidity using saturated salt solutions.

In certain embodiments, the pH dependent shell compositions described herein may have a burst strength ranging from any of about 50 kg, about 60 kg, about 70 kg, about 80 kg, or about 90 kg to any of about 100 kg, about 110 kg, about 120 kg, about 130 kg, about 140 kg, or about 150 kg. Burst strength, which is an indication of robustness of capsules, is determined using a texture analyzer. The texture analyzer compressed the capsule until the capsule burst. The force, in kilograms, required to make the capsule burst is defined as burst strength.

In an embodiment, the pH dependent shell composition and the pH dependent softgel capsule may be free or substantially free of conventional pH dependent polymers and/or be free of a pH dependent overcoat over the softgel shell.

In an embodiment, the pH dependent shell composition and the pH dependent softgel capsule may include divalent cation salts, such as Ca⁺⁺ (e.g., CaCl₂) or Mg⁺⁺ (e.g., MgCl₂). In another embodiment, the pH dependent shell composition and the pH dependent softgel capsule may be free or substantially free of divalent cation salts, such as Ca⁺⁺ (e.g., CaCl₂) or Mg⁺⁺ (e.g., MgCl₂). In a further embodiment, the pH dependent shell composition may not include the step of the addition of divalent cation salts, such as Ca⁺⁺ (e.g., CaCl₂) or Mg⁺⁺ (e.g., MgCl₂) other than an amount of divalent cation salts that may be present in other components.

In an embodiment, the pH dependent shell composition may optionally comprise additional agents, such as stabilizers or binders (e.g., gellan gum), coloring agents, flavorings agents, sweetening agents, fillers, antioxidants, diluents, pH modifiers or other pharmaceutically acceptable excipients or additives such as synthetic dyes and mineral oxides.

Exemplary suitable coloring agents for the fill composition and for the shell composition may include, but not be limited to, colors such as e.g., white, black, yellow, blue, green, pink, red, orange, violet, indigo, and brown. In specific embodiments, the color of the dosage form can indicate the contents (e.g., one or more active ingredients) contained therein.

Exemplary suitable flavoring agents for the fill composition and for the shell composition may include, but not be limited to, “flavor extract” obtained by extracting a part of a raw material, e.g., animal or plant material, often by using a solvent such as ethanol or water; natural essences obtained by extracting essential oils from the blossoms, fruit, roots, etc., or from the whole plants.

Additional exemplary flavoring agents for the fill composition and for the shell composition may include, but not be limited to, breath freshening compounds like menthol, spearmint, and cinnamon, coffee beans, other flavors or fragrances such as fruit flavors (e.g., cherry, orange, grape, etc.), especially those used for oral hygiene, as well as actives used in dental and oral cleansing such as quaternary ammonium bases. The effect of flavors may be enhanced using flavor enhancers like tartaric acid, citric acid, vanillin, or the like.

Exemplary sweetening agents for the fill composition and for the shell compositions may include, but not be limited to, one or more artificial sweeteners, one or more natural sweeteners, or a combination thereof. Artificial sweeteners include, e.g., acesulfame and its various salts such as the potassium salt (available as Sunett®), alitame, aspartame (available as NutraSweet® and Equal®), salt of aspartame-acesulfame (available as Twinsweet®), neohesperidin dihydrochalcone, naringin dihydrochalcone, dihydrochalcone compounds, neotame, sodium cyclamate, saccharin and its various salts such as the sodium salt (available as Sweet'N Low®), stevia, chloro derivatives of sucrose such as sucralose (available as Kaltame® and Splenda®), and mogrosides. Natural sweeteners include, e.g., glucose, dextrose, invert sugar, fructose, sucrose, glycyrrhizin; monoammonium glycyrrhizinate (sold under the trade name MagnaSweet®); Stevia rebaudiana (Stevioside), natural intensive sweeteners, such as Lo Han Kuo, polyols such as sorbitol, mannitol, xylitol, erythritol, and the like.

In some embodiments, the pH dependent shell composition and/or the pH dependent softgel capsule may be tested in a disintegration/dissolution test performed in a USP Apparatus II with paddles at a speed of 50 rpm in acidic medium of 0.1N HCl (pH 1.2 optionally with Pepsin) followed by buffer medium (pH 6.8 phosphate buffer optionally with Pancreatin). The pH dependent shell composition according to this embodiment may remain intact for at least about 10 minutes, at least about 15 minutes, at least about 30 minutes, at least about one hour, at least about two hours, at least about three hours, at least about four hours, or at least about five hours, in acidic medium and may disintegrate in buffer medium in about 60 minutes or less, in about 45 minutes or less, in about 30 minutes or less, in about 20 minutes or less, in about 15 minutes or less, in about 10 minutes or less, or in about 5 minutes or less.

While the buffer medium of the two stage dissolution/disintegration test has a pH 6.8, it should be noted that a similar dissolution/disintegration profile may be attained at a buffer medium having a pH of about 3.5 or higher (optionally with Pancreatin). It should also be noted that the presence of Pepsin (in acidic medium) and Pancreatin (in buffer medium) are not necessitated by pharmacopeial methods but are used herein in certain instances to simulate more aggressive environments that better mimic in-vivo conditions.

In certain embodiments, the pH dependent shell composition described herein remains intact in acidic environment (e.g., stomach environment or simulated stomach environment such as simulated gastric fluid, 0.1N HCl optionally with Pepsin) for at least about 10 minutes, at least about 15 minutes, at least about 30 minutes, at least about one hour, at least about two hours, at least about three hours, at least about four hours, or at least about five hours but ruptures/dissolves/disintegrates in a pH of about 3.5 or higher (e.g., in the duodenal area and/or in the intestines or simulated environments thereof such as pH 6.8 buffer medium optionally with Pancreatin) in up to about 5 minutes, up to about 10 minutes, up to about 15 minutes, up to about 20 minutes, up to about 25 minutes, up to about 30 minutes, up to about 35 minutes, up to about 40 minutes, up to about 45 minutes, or up to about 60 minutes.

In an embodiment, the pH dependent shell composition comprises: (a) gelatin, (b) dextrose, (c) a pH dependent polymer (e.g., pectin such as a low methoxyl pectin), (d) a plasticizer (e.g., glycerin, sorbitol, and combinations thereof), and optionally (e) a stabilizer and/or binder (e.g., gellan gum). The amounts and wt:wt ratios of these components may be in accordance with any of the values or ranges described hereinabove.

In an embodiment, the pH dependent shell composition consists essentially of: (a) a gelatin, (b) dextrose, (c) a pH dependent polymer (e.g., pectin such as a low methoxy pectin), (d) a plasticizer (e.g., glycerin, sorbitol, gellan gum, and combinations thereof), and optionally (e) a stabilizer and/or binder (e.g., gellan gum). The amounts and wt:wt ratios of these components may be in accordance with any of the values or ranges described hereinabove.

In an embodiment, the pH dependent shell composition consists of: (a) a gelatin, (b) dextrose, (c) a pH dependent polymer (e.g., pectin such as a low methoxyl pectin), (d) a plasticizer (e.g., glycerin, sorbitol, gellan gum, and combinations thereof), and optionally (e) a stabilizer and/or binder (e.g., gellan gum). The amounts and wt:wt ratios of these components may be in accordance with any of the values or ranges described hereinabove.

In an embodiment, the pH dependent shell composition comprises/consists essentially of/consists of: (a) about 25 wt. % to about 85 wt. %, from about 25 wt. % to about 80 wt. % from about 30 wt. % to about 85 wt. %, from about 30 wt. % to about 75 wt. %, from about 35 wt. % to about 70 wt. %, from about 30 wt. % to about 65 wt. %, from about 40 wt. % to about 65 wt. %, from about 30 wt. % to about 55 wt. %, from about 30 wt. % to about 40 45 wt. %, about 40 wt. % to about 80 wt. %, from about 45 wt. % to about 65 wt. %, from about wt. % to about 60 wt. %, from about 45 wt. % to about 75 wt. %, or from about 50 wt. % to about 70 wt. % gelatin, (b) from about 0.001 wt. % to about 1.0 wt. %, from about 0.002 wt. % to about 0.008 wt. %, from about 0.005 wt. % or about 0.01 wt. % to about 4 wt. %, from about 0.1 wt. % or about 0.15 wt. % to about 3 wt. %, from about 0.1 wt. % to about 1 wt. %, from about 0.1 or about 0.15 wt. % or about 0.2 wt. % or about 0.25 wt. % to about 2 wt. %, from about 0.1 wt. % to about 0.2 wt. %, from about 0.1 wt. % to about 0.4 wt. %, from about 0.05 wt. % to about 0.5 wt. % dextrose, (c) about 2 wt. % to about 20 wt. %, from about 3 wt. % to about 15 wt. %, from about 3 wt. % to about 18 wt. %, from about 5 wt. % to about 15 wt. %, from about 3 wt. % to about 5.5 wt. %, from about 3.5 wt. % to about 6.5 wt. %, from about 2.5 wt. % to about 7 wt. %, from about 4 wt. % to about 11 wt. %, from about 7 wt. % to about 12 wt. %, from about 8 wt. % to about 13 wt. %, or from about 5 wt. % to about 10 wt. % of a pH dependent polymer (e.g., pectin such as a low methoxy pectin), (d) about 10 wt. % to about 40 wt. %, from about 15 wt. % to about 35 wt. %, from about 15 wt. % to about 45 wt. %, from about 15 wt. % to about 40 wt. %, from about 18 wt. % to about 45 wt. %, from about 18 wt. % to about 42 wt. %, from about 20 wt. % to about 35 wt. %, from about 20 wt. % to about 30 wt. %, from about 25 wt. % to about 30 wt. % of a plasticizer, and optionally (e) about 0.05 wt. % to about 5 wt. %, about 0.1 wt. % to about 3 wt. %, about 0.1 wt. % to about 2 wt. %, or about 0.2 wt. % to about 2 wt. % of stabilizer and/or binder (e.g., gellan gum). All wt. % being based on the total weight of the dry pH dependent shell composition.

### Process of Preparing a Modified Release Softgel Capsule

In certain embodiments, the instant disclosure encompasses a process for preparing any of the modified release softgel capsules described herein, as illustrated in FIG. 5. The process 500 may include encapsulating any of the controlled release fill compositions described herein inside any of the pH dependent shell compositions described herein (520). Encapsulation of the fill composition can be accomplished in any conventional manner. As an example, a rotary die encapsulation may be used.

In certain embodiments, after encapsulation, the modified release softgel capsules may be dried (530). Thereafter, the softgel capsules may be annealed (which may also be referred to as cured) at an annealing (or curing) temperature for an annealing (or curing) duration (540) to prepare the final modified release softgel capsules (550).

The annealing temperature may range from about 25° C. to about 80° C., from about 30° C. to about 70° C., or from about 40° C. to about 60° C., from about 25° C. to about 55° C., from about 25° C. to about 50° C., from about 30° C. to about 60° C., or from about 35° C. to about 50° C. If annealing occurs, the annealing temperature should be high enough to enhance the delayed release properties of the pH dependent shell composition and to facilitate matrix formation inside the pH dependent shell composition, but not so high that it would melt or degrade the softgel capsule or any component of the pH dependent shell composition or of the controlled release fill composition.

The annealing duration may range from about 10 minutes to about 24 hours, from about 30 minutes to about 12 hours, from about 45 minutes to about 5 hours, or from about 60 minutes to about 3 hours. In some embodiments, the annealing duration may range from about 12 hours to about 168 hours, from about 18 hours to about 120 hours, from about 24 hours to about 72 hours, about 24 hours, about 48 hours, about 72 hours, or any sub-range or single values therein.

In an embodiment, the annealing of the softgel capsule may be performed at a temperature of about 40° C. for about 24 hours. In an embodiment, the annealing of the softgel capsule may be performed at a temperature of about 40° C. for about 48 hours. In an embodiment, the annealing of the softgel capsule may be performed at a temperature of about 40° C. for about 72 hours. In an embodiment, the annealing of the softgel capsule may be performed at a temperature of about 60° C. for about 1-3 hours. In an embodiment, the annealing of the softgel capsule may be performed at a temperature of about 65° C. for about 90 minutes.

In certain embodiments, the curing may occur in air (without any particular controls as to the content of nitrogen or oxygen or humidity). In certain embodiments, the annealing may occur under inert conditions (e.g., in nitrogen).

In certain embodiments, prior to encapsulation, process 500 may include preparation of the controlled release fill composition. The controlled release fill composition may be prepared by combining, e.g., mixing, at least one active agent with the controlled release material and optionally with a hydrophilic carrier, if one is present. Any other ingredients, such as pharmaceutically acceptable excipients, may be also mixed into the mixture to form the controlled release fill composition. For example, in process 500 in FIG. 5, the active agent (referred to as drug substance 512) is mixed with a controlled release material (exemplified to as polyethylene oxide 514), a hydrophilic carrier (exemplified as polyethylene glycol 516), and other ingredients (518).

In certain embodiments, prior to encapsulation, process 500 may include preparation of the pH dependent shell composition (not shown in FIG. 5). The pH dependent softgel capsule may be prepared by, for example, mixing gelatin, dextrose, pectin, optionally a plasticizer, optionally a binder (such as gellan gum), and optionally any other ingredients, e.g., pharmaceutically acceptable excipients. In preferred embodiments, the pH dependent shell composition is free of additional pH dependent polymers (such as conventional pH dependent synthetic polymers) and those may not be added to the mixture when preparing a pH dependent shell composition according to certain embodiments.

The ribbon thickness of the pH dependent shell composition (as used for example during rotary die encapsulation) may also be tuned to control the pH dependent dissolution profile of the final pH dependent shell composition and to facilitate targeted release of the fill composition to specific areas within the GIT. The ribbon thickness of the pH dependent shell composition may range, without limitations, from any of about 0.02 inches, about 0.022 inches, about 0.024 inches, about 0.026 inches, about 0.028 inches, or about 0.030 inches to any of about 0.032 inches, about 0.034 inches, about 0.036 inches, about 0.038 inches, about 0.04 inches, about 0.042 inches, about 0.044 inches, or about 0.050 inches or any sub-range or single value therein.

In an embodiment, the process for preparing a pH dependent softgel capsule comprises, consists essentially of, or consists of a) preparing any of the controlled release fill compositions described herein; b) encapsulating the controlled release fill composition from step a) in any of the pH dependent shell compositions described herein (e.g., via rotary die encapsulation); c) drying the encapsulated pH dependent softgel capsules (e.g., by tumble drying or regular drying in a basket without tumbling); and optionally d) curing/annealing the pH dependent softgel capsule in accordance with any of the curing/annealing conditions described herein.

In certain embodiments, drying is performed at about 10° C. to about 50° C., about 15° C. to about 40° C., or about 20° C. to about 35° C. at a relative humidity of about 5% to about 40%, about 10% to about 30%, or about 15% to about 25%.

In certain embodiments, reference to drying and curing/annealing should be distinguished here. The purpose of drying the modified release softgel capsules described herein is to remove excess water from the modified release softgel capsule immediately after encapsulation. So, the capsules will be physically stable. The purpose of curing/annealing the modified release softgel capsules described herein is to enhance the delayed release property of the modified release softgel capsule. Hence, the presence of a drying step is not the same as a curing/annealing step and similarly the presence of a curing/annealing step is not the same as a drying step.

### Stability of Modified Release Softgel Capsules

In certain embodiments, the pH dependent shell compositions described herein are chemically and physically stable.

For instance, their chemical stability may be evidenced by the content of the active agent in the fill composition (e.g., content of fish oil constituents when the fill composition includes fish oil). In certain embodiments, the content of the fill composition constituents is substantially similar (or within specifications), after storage for up to 12 months, up to 6 months, up to 3 months, or up to 1 months (at ambient conditions or at stressed conditions of 40° C. and 75% relative humidity for any of these durations) as compared to the raw composition before storage.

In certain embodiments, the physical stability of the modified release softgel capsules and of the pH dependent shell composition may be evidenced by the dissolution profile of the capsule in acidic medium and in buffer medium. For instance, the dissolution profile of the capsule in acidic medium and in buffer medium is substantially similar (or within specifications), after storage for up to 12 months, up to 6 months, up to 3 months, or up to 1 months (at ambient conditions or at stressed conditions of 40° C. and 75% relative humidity for any of these durations) as compared to the dissolution profile of the capsule before storage.

The term “substantially similar” may refer to a particular value being within about 30%, within about 25%, within about 20%, within about 15%, within about 10%, within about 5%, or within about 1% of a corresponding comparative value. The percentage being calculated based on the face value of the comparative value. For instance, a dissolution time range of 27 minutes to 33 minutes may be considered within 10% of comparative dissolution time of 30 minutes.

In certain embodiments, the instant disclosure may also be directed to a method of stabilizing any of the modified release softgel capsules described herein. The method may include protecting (e.g., from oxidation or another potential source of chemical degradation) any of the fill compositions described herein by encapsulating any of the fill compositions described herein (including at least one active agent) in any of the pH dependent shell compositions described herein.

In certain embodiments, the pH dependent shell composition described herein produce a robust modified release softgel capsule that has little or no premature release of the fill composition in acidic environment (e.g., stomach environment). For instance, modified release softgel capsules described herein may release up to about 10 wt. %, up to about 9 wt. %, up to about 8 wt. %, up to about 7 wt. %, up to about 6 wt. %, up to about 5 wt. %, up to about 4 wt. %, up to about 3 wt. %, up to about 1 wt. %, or 0 wt. %, of the fill composition based on total weight of the fill composition in acidic environment after exposure to the acidic environment for up to about 150 minutes, up to about 120 minutes, up to about 105 minutes, up to about 90 minutes, up to about 75 minutes, up to about 60 minutes, up to about 45 minutes, up to about 30 minutes, up to about 15 minutes, up to about 10 minutes, or up to about 5 minutes. The release time of the fill composition should not be confused with the release time of the active agent in the fill composition as the release profile of the active agent from the fill composition would be dictated by the constituents of the controlled release fill composition, as described in detail in the controlled release fill composition section.

In certain embodiments, curing/annealing modified release softgel capsules described herein (i.e., ones that are encapsulated with a pH dependent shell composition) may reduce or eliminate the number of capsules exhibiting any amount of premature release in acidic environment. For instance, the number of cured/annealed capsules exhibiting premature release in acidic environment (after exposure to the acidic environment for up to about 150 minutes, up to about 120 minutes, up to about 105 minutes, up to about 90 minutes, up to about 75 minutes, up to about 60 minutes, up to about 45 minutes, up to about 30 minutes, up to about 15 minutes, up to about 10 minutes, or up to about 5 minutes) may be up to about 30%, up to about 25%, up to about 20%, up to about 15%, up to about 10%, up to about 5%, up to about 3%, up to about 2%, up to about 1%, or 0% of the total number of capsules in the batch.

In comparison, without curing/annealing, the number of capsules (having the same composition) exhibiting premature release in acidic environment (after exposure to the acidic environment for up to about 150 minutes, up to about 120 minutes, up to about 105 minutes, up to about 90 minutes, up to about 75 minutes, up to about 60 minutes, up to about 45 minutes, up to about 30 minutes, up to about 15 minutes, up to about 10 minutes, or up to about 5 minutes) may be greater than about 2%, greater than about 5%, greater than about 10%, greater than about 15%, greater than about 20%, greater than about 30%, greater than about 40%, greater than about 50%, greater than about 60%, greater than about 70%, greater than about 80%, or greater than about 90% of the total number of capsules in the batch.

In certain embodiments, curing/annealing modified release softgel capsules described herein (i.e., ones that are encapsulated with a pH dependent shell composition) may reduce or eliminate the amount of fill composition released from capsules that exhibit some premature release in acidic environment (e.g., after exposure to the acidic environment for up to about 150 minutes, up to about 120 minutes, up to about 105 minutes, up to about 90 minutes, up to about 75 minutes, up to about 60 minutes, up to about 45 minutes, up to about 30 minutes, up to about 15 minutes, up to about 10 minutes, or up to about 5 minutes).

For instance, the amount of fill composition released from cured/annealed capsules exhibiting some premature release in acidic environment (e.g., after exposure to the acidic environment for up to about 150 minutes, up to about 120 minutes, up to about 105 minutes, up to about 90 minutes, up to about 75 minutes, up to about 60 minutes, up to about 45 minutes, up to about 30 minutes, up to about 15 minutes, up to about 10 minutes, or up to about 5 minutes) may be up to about 5 wt. %, up to about 4 wt. %, up to about 3 wt. %, up to about 2 wt. %, up to about 1 wt. %, or 0% of the total weight of fill composition in the capsule.

In comparison, without curing, the amount of fill composition released from capsules (having the same composition) exhibiting premature release in acidic environment (e.g., after exposure to the acidic environment for up to about 150 minutes, up to about 120 minutes, up to about 105 minutes, up to about 90 minutes, up to about 75 minutes, up to about 60 minutes, up to about 45 minutes, up to about 30 minutes, up to about 15 minutes, up to about 10 minutes, or up to about 5 minutes) may be greater than about 1 wt. %, greater than about 2 wt. %, greater than about 3 wt. %, greater than about 4 wt. %, greater than about 5 wt. %, greater than about 6 wt. %, greater than about 7 wt. %, greater than about 8 wt. %, greater than about 9 wt. %, greater than about 10 wt. %, greater than about 15 wt. %, or greater than about 20 wt. % of the total weight of fill composition in the capsule.

### Dual Controlled Release Softgel Capsule

The softgel capsules described herein include two levels of controlled release. The first level being dictated by the pH dependent shell composition, which facilitates targeted release of the fill composition in specific areas within the GIT. The second level being dictated by the controlled release fill composition, which facilitates a controlled release profile for the active agent at the specific area within the GIT.

In certain embodiments, the instant disclosure is directed to a method for tuning/modulating/controlling the dissolution location and dissolution profile of any of the delayed release capsules described herein (i.e., comprises of a controlled release fill material encapsulated in a pH dependent shell composition). The method includes adjusting at least one of i)-iv) to control dissolution location of the pH dependent shell composition along the gastrointestinal tract of a subject: i) amounts of at least one of pectin, gelatin, dextrose, and plasticizer in the pH dependent shell composition; ii) annealing temperature of the modified release softgel capsule; iii) annealing duration of the modified release softgel capsule; or iv) ribbon thickness of the pH dependent shell composition. The method further includes adjusting at least one of (v)-(vii) to attain a target dissolution profile of the at least one active agent: v) amount of a controlled release material in the controlled release fill composition, or vi) annealing temperature of the modified release softgel capsule; or vii) annealing duration of the modified release softgel capsule.

In certain embodiments, the instant disclosure also encompasses methods of treating a condition, treatable by any of the modified release softgel capsules described herein, by administering to a subject in need thereof any of the modified release softgel capsules described herein.

### EXAMPLES

Specific embodiments of the disclosure will now be demonstrated by reference to the following examples. It should be understood that these examples are disclosed solely by way of illustrating the disclosure and should not be taken in any way to limit the scope of the present disclosure.

### Example 1—Effect of Dextrose Concentration on Manufacturing of pH Dependent Shell Composition

PH dependent shell compositions with varying concentrations of dextrose were prepared to study the effect of the dextrose concentration on the manufacturability of the composition. The pH dependent shell compositions are set forth in Table 1.

TABLE 1
Dry Shell Compositions

| | Group | Group | Group | Group | Group |
|---|---|---|---|---|---|
| | No. 1 | No. 2 | No. 3 | No. 4 | No. 5 |
| Ingredient | wt. % | wt. % | wt. % | wt. % | wt. % |
| Pectin | 8-12 | 7-11 | 7-12 | 8-13 | 6-9 |
| Gelatin | 45-65 | 38-58 | 38-58 | 38-58 | 38-58 |
| Glycerin | 28-45 | 25-35 | 25-35 | 25-35 | 25-35 |
| Water | 8-15 | 6-15 | 6-15 | 6-15 | 6-15 |
| Dextrose | 0.02-0.10 | 0.01-0.06 | 0.10-0.20 | 0.10-0.30 | None |
| Total | 100 | 100 | 100 | 100 | 100 |

The effect of varying amounts of dextrose in the pH dependent shell composition on rupture time at pH 6.8 is in Table 2.

TABLE 2

| | | Dissolution Results | | Dissolution Results | |
|---|---|---|---|---|---|
| | | at T = 0 | | at T = 6 months | |
| | | Acid | | Acid | |
| | | Stage | Buffer | Stage | Buffer |
| Group | Dextrose | (0.1N | Stage | (0.1N | Stage |
| No. | (wt. %) | HCl) | (pH 6.8) | HCl) | (pH 6.8) |
| 1 | 0.01 | Pass | Pass | Pass | Ruptured |
| | | (Intact for | (Ruptured | (Intact for | in |
| | | 2 hrs) | in 8 Min) | 2 hrs) | 25 minutes |
| 2 | 0.05 | Pass | Pass | Pass | No rupture |
| | | (Intact for | (Ruptured | (Intact for | for |
| | | 2 hrs) | in 4 Min) | 2 hrs) | 60 minutes |
| 3 | 0.1 | Pass | Pass | Pass | No rupture |
| | | (Intact for | (Ruptured | (Intact for | for |
| | | 2 hrs) | in 3 Min) | 2 hrs) | 60 minutes |
| 4 | 0.15 | Pass | Pass | Pass | No rupture |
| | | (Intact for | (Raptured | (Intact for | for |
| | | 2 hrs) | in11 Min) | 2 hrs) | 60 minutes |
| 5 | None | Failed | — | Pass | Ruptured |
| | | (Ruptured | | (Intact for | in |
| | | in | | 2 hrs) | 28 minutes |
| | | 90 minutes) | | | |

Dextrose is a reducing sugar and is believed to interact with gelatin by causing the gelatin to cross-link. When gelatin is crosslinked, its solubility is reduced. It was shown that dextrose stabilized (i.e., reduced leakage) of the pectin softgel capsule in acidic medium.

### Example 2—Effect of Curing on Capsule Release Properties

pH dependent shell compositions were prepared to study the effect of curing on the release properties of the capsules, as it related to the fill composition in general (note that the release profile of the active agent from the fill composition is a second level of controlled release which was not exemplified in this particular example). The pH dependent shell compositions are set forth in Table 3.

TABLE 3
Gel Mass Formulations in wt. % in Dry Capsule Shell

| Ingredient | Lot 1 | Lot 2 | Lot 3 |
|---|---|---|---|
| Non-amidated pectin | 7.0-12.0 | 8.0-12.0 | 8.0-12.0 |
| Dextrose | 0.02-0.10 | 0.10-1.0 | 0.10-1.0 |
| Glycerin | 28-45 | 28-45 | 28-45 |
| Gelatin | 45-65 | 45-65 | 45-65 |
| Water | 8-15 | 8-15 | 8-15 |
| Total | 100 | 100 | 100 |
| Additional Properties | | | |
| Weight non-amidated pectin | 1:7 | 1:7.5 | 1:7.5 |
| to weight gelatin ratio | | | |
| weight glycerin to weight | 1:2 | 1:2 | 1:2 |
| gelatin ratio | | | |
| Gel mass viscosity (cPs) | 115,000 | 121,000 | 121,000 |
| % Capsules having | 67% | 42% | 50% |
| Premature Release | | | |
| Prior to Curing | | | |

Existing commercial products exhibit premature release in a large number of capsules, increased amounts of fill composition prematurely released, and in some instances almost a 100 wt. % of the fill composition being released in acidic medium within a 10 minute duration.

Coated softgel capsules were contemplated but those did not dissolve in buffer medium for an extended duration (longer than about 60 minutes and in some instances as long as 120 minutes). The long dissolution in buffer medium was believed to suggest that coated softgel capsules would not be bioavailable. This along with the challenge of two step manufacturing process encouraged exploration of pH dependent shell compositions to form a modified release softgel capsule without a separate coating.

The pH dependent shell compositions set forth in Table 3 were used to form pectin softgels which reduced the occurrence of premature release and the amount of fill composition that is prematurely released to a certain extent (as compared to existing commercial products).

However, prior to curing, a significant fraction of the softgel capsules in each lot still continued to exhibit some premature release of the fill composition in acidic environment (e.g., 0.1N HCl), as summarized in Table 3 in the “% capsules having premature release prior to curing.” About 60 to about 72 capsules were tested from each lot to assess the % capsules having premature release prior to curing.

In certain embodiments, about 10 wt. % of the fill composition was released from capsules having premature release, prior to curing. In certain embodiments, more than 10 wt. % of the fill composition or less than 10 wt. % of the fill composition was released from capsules having premature release, prior to curing.

As will be shown in subsequent examples, curing reduced the occurrence of premature release, the amount of fill composition released upon occurrence of premature release, and in some instances eliminated premature release altogether.

The pectin softgel capsules were cured to enhance their stability in acidic environment (e.g., 0.1N HCl). The pectin softgels were packaged in cartons (for bulk) or in high density polyethylene (HDPE) bottles and placed into an oven heated to 40° C. No humidity controls were used. The only variable across samples was the curing time. The curing study results of lots 1, 2, and 3 are summarized in Table 4 below.

TABLE 4
Results of Curing Studies

| | Prior to | | Dissolution After Curing | | |
|---|---|---|---|---|---|
| | Curing | | | 0.1N HCl | |
| | % | | | Number Capsules | |
| | Capsules | | | having Premature | pH 6.8 |
| | having | No of | | Release (% from | buffer |
| Sam- | Premature | Capsules | Curing | total capsules | (Rupture |
| ple | Release | Tested | Time | tested) | Time) |
| Lot 1 | 67% | 12 | 24 hours | 3 (25%) | 8 minutes |
| | | 36 | 48 hours | None | 7 minutes |
| Lot 2 | 42% | 60 | 48 hours | 1 (1.7%) | 7 minutes |
| | | 72 | 72 hours | None | 9 minutes |
| Lot 3 | 50% | 60 | 48 hours | None | 7 minutes |
| | | 60 | 72 hours | None | 7 minutes |

The dissolution of the pH dependent shell composition after curing was assessed in accordance with the USP enteric testing method for a two stage enteric dissolution test applicable to uncoated enteric softgels. Unless specified otherwise, the acidic medium, buffer medium, apparatus, and dissolution test conditions for all dissolution/disintegration/rupture results and/or properties throughout this application were as described herein with respect to the two stage enteric dissolution test.

A USP Apparatus II with paddles was used, at a paddle speed of 50 rpm at 37° C. The acidic stage medium was 0.1N HCl. The buffer stage medium was pH 6.8 phosphate buffer. For vitamin mineral supplements and/or nutraceutical products, enteric capsules should remain intact for at least 60 minutes in acidic medium to pass the first stage and rupture within 45 minutes in buffer stage medium to pass the second stage. For pharmaceutical products, enteric capsules should remain intact for at least 120 minutes in acidic medium to pass the first stage and rupture within 45 minutes in buffer stage medium to pass the second stage.

Curing of the softgel capsules was assessed at 24 hours, 48 hours, 72 hours, 120 hours, 168 hours, and 288 hours. Although only data up to 72 hours is presented herein.

Table 5 depicts the amount of premature release of fill composition from pectin softgel capsules of lot 3, prior to curing and after curing, in acidic medium following a USP enteric test criteria at the end of 2 hours. The maximum amount of fill composition that was released was 5%. The pectin softgel capsules in lot 3 included fish oil (which includes docosahexaenoic acid (DHA) and eicosapentaenoic acid (EPA)) in the fill composition.

TABLE 5
Lot 3 - Amount of Fill composition Prematurely Released in
Acidic Media (0.1N HCl) Following USP Enteric Test Criteria

| | Dissolution in 0.1N HCl | | Dissolution in 0.1N HCl | |
|---|---|---|---|---|
| | after 2 hours, | | after 2 hours, | |
| Vessel | Prior to Curing | | After Curing for 72 hours | |
| No. | % EPA | % DHA | % EPA | % DHA |
| V1 | 5 | 4 | 0 | 0 |
| V2 | 3 | 3 | 0 | 0 |
| V3 | 3 | 3 | 0 | 0 |
| V4 | 3 | 3 | 0 | 0 |
| V5 | 1 | 1 | 0 | 0 |
| V6 | 3 | 3 | 0 | 0 |

The curing data showed that curing significantly reduced or eliminated premature release of fill composition from pectin capsules in acidic medium resulting in capsules with robust enteric properties and high quality enteric product.

Note that all of the pectin softgel capsules tested in Table 5 dissolved in pH 6.8 buffer within 15 minutes.

### Example 3—Enteric Dissolution Data in Simulated Gastric Fluid (SGF) with Pepsin

Cured pectin capsules, having the gel mass formulas summarized in Table 6A, were subjected to an enteric rupture testing using SGF (0.1N HCl) with pepsin (to simulate in-vivo conditions in humans) for two stage enteric dissolution studies.

TABLE 6A
Gel Mass Formulations in wt. % in Dry Capsule Shell

| Ingredient | Lot 4 | Lot 5 |
|---|---|---|
| Non-amidated pectin | 7.0-11.0 | 8.0-13.0 |
| Dextrose | 0.02-0.08 | 0.02-0.08 |
| Glycerin | 18-42 | 18-42 |
| Gelatin | 45-65 | 45-65 |
| Water | 8-15 | 8-15 |
| Total | 100 | 100 |

TABLE 6B
Dissolution of Pectin Softgel Capsules from Table
6A in Acidic Medium with and without Pepsin

| Lot No | 0.1N HCl | 0.1N HCl with Pepsin |
|---|---|---|
| Lot 4 | Intact for 120 minutes | Intact for 120 minutes |
| Lot 5 | Intact for 120 minutes | Intact for 120 minutes |

Pepsin did not affect the dissolution of pectin shells in 0.1N HCl medium when an appropriate shell composition, e.g., Gelatin to Pectin ratio is used. In lots 4 and 5, illustrated in Tables 6A and 6B, the gelatin to pectin w:w ratio was 7:1. Without being construed as limiting, it is believed that the pectin softgels are robust and that the gelatin-pectin networks are strong enough to withstand the effect of pepsin as evidenced by the pectin softgel capsules remaining intact for 120 minutes in 0.1N HCl even in the presence of pepsin which represents a Biorelevant media unlike the Pharmacopeial method which doesn't include enzyme. Hence, it is believed that the pectin softgel capsules will also be sufficiently robust in-vivo.

### Example 4—Modulation of Pectin Capsule Rupture Time in Enteric Media by Changing the Gelatin to Pectin Ratio

Pectin softgel capsules were prepared with varying Gelatin to Pectin ratio. The composition of the various lots is summarized in Table 7B below. The rupture time of the pectin capsules in SGF (0.1N HCl) with pepsin varied with varying Gelatin to Pectin ratio, as summarized in Table 7A below.

TABLE 7A

| Lot No | Gelatin-Pectin ratio | 0.1N HCl with Pepsin |
|---|---|---|
| Lot 6 | 18:1 | Ruptured at 12 minutes |
| Lot 7 | 12:1 | Ruptured at 36 minutes |
| Lot 8 | 8:1 | Intact for 120 minutes |
| Lot 1 | 7:1 | Intact for 120 minutes |

TABLE 7B
Gel Mass Formulas Based on Dry Shell Composition
for Gelatin-Pectin Ratio Study (wt. %)

| Ingredient | Lot 6 | Lot 7 | Lot 8 | Lot 1 |
|---|---|---|---|---|
| Pectin | Non- | Amidated | Non- | Non- |
| | amidated | pectin | amidated | amidated |
| | pectin | 6.0-10.0 | pectin | pectin |
| | 3.0-8.0 | | 8.0-15.0 | 7.0-12.0 |
| Dextrose | 0 | 0 | 0.02-0.10 | 0.02-0.10 |
| Glycerin | 8-15 | 21-41 | 8-15 | 28-45 |
| Sorbitol | 21-32 | 0 | 21-32 | 0 |
| Gelatin | 44-65 | 42-61 | 44-65 | 45-65 |
| Water | 8-15 | 8-15 | 8-15 | 8-15 |
| Total | 100.0 | 100.0 | 100.0 | 100.0 |

All of the pectin softgel capsules from Table 7A ruptured in pH 6.8 buffer within 45 minutes. Table 7A illustrates that the rupture time of the pectin softgel capsules in acidic medium may be modulated by changing the gelatin to pectin ratio.

### Example 5—Effect of Softgel Ribbon Thickness on the Enteric Performance of the Pectin Softgel Capsule

Pectin softgel capsules were prepared with varying ribbon thicknesses. The compositions of the dry pH dependent shell composition for lots manufactured with varying ribbon thicknesses are summarized in Table 8A below. The dissolution of the pectin capsules of varying ribbon thickness, after curing for about 72 to 96 hours, in SGF (0.1N HCl) and in pH 6.8 buffer was evaluated. The results are summarized in Table 8B below.

TABLE 8A
Gel Mass Formulas Based on Dry Shell Composition for Ribbon Thickness
Study (wt. %)

| Ingredient | Lot 9 | Lot 10 | Lot 11 | Lot 12 | Lot 13 | Lot 14 |
|---|---|---|---|---|---|---|
| Pectin | Amidated | Non- | Amidated | Non- | Non- | Non- |
| | pectin | amidated | pectin | amidated | amidated | amidated |
| | 6.5-10.0 | pectin | 6.5-10.0 | pectin | pectin | pectin |
| | | 8.0-12.0 | | 7.0-11.0 | 8.0-12.0 | 8.0-13.0 |
| Dextrose | None | 0.020-0.15 | None | 0.02-0.06 | 0.020-0.15 | 0.02-0.10 |
| Glycerin | 22-40 | 21-41 | 22-40 | 18-42 | 21-41 | 18-42 |
| Gelatin | 42-58 | 44-61 | 42-58 | 45-65 | 44-61 | 45-65 |
| Water | 8-15 | 8-15 | 8-15 | 8-15 | 8-15 | 8-15 |
| Total | 100.0 | 100.0 | 100.0 | 100.0 | 100.0 | 100.0 |

TABLE 8B
Dissolution of Cured Softgel Pectin Capsules
with Varying Ribbon Thickness

Dissolution on Cured Pectin
Softgel Capsules

| | Ribbon | | pH 6.8 Buffer |
|---|---|---|---|
| | Thickness | 0.1N HCl | (Rupture |
| Lot No | (inches) | (120 minutes) | Time, min) |
| Lot 9 | 0.028 | Intact | 7 |
| Lot 10 | 0.030 | Intact | 1 |
| Lot 11 | 0.032 | Intact | 8 |
| Lot 12 | 0.034 | Intact | 5 |
| Lot 13 | 0.036 | Intact | 3 |
| Lot 14 | 0.038 | Intact | 7 |

The dissolution results depicted in Table 8B illustrate that pectin softgel capsules, having a ribbon thickness ranging from 0.028 inches to 0.038 inches, after curing, were shown to be robust and were shown to meet the enteric criteria for pharmaceutical products and for VMS (vitamin, mineral, supplements) products. This thickness range should not be construed as limiting. In certain embodiments, thicker ribbons or thinner ribbons may also be utilized.

### Example 6—pH Dependent Shell Composition Viscosity Upon Aging

Pectin and gelatin interact with each other to form networks which contribute to significant increases in gel mass viscosity shown in FIG. 1. The interaction between pectin and gelatin is believed to contribute to the capsule shell composition's delayed release properties. However, as seen in FIG. 1, the viscosity of gel mass of the pH dependent shell composition decreases over time. The viscosities and % reduction are summarized in Table 9 below.

The viscosity in this example and throughout the description was measured using a rheometer (Rheostress 6000 by Thermo Fisher) at 60° C. The tests were performed at ambient conditions. The gel mass samples were loaded onto the platform of the rheometer, which was maintained at 60° C. A 40 mm disc oscillated at a frequency of 0.1 Hz to provide a fixed shear rate. The viscosity was obtained by measuring the shear stress and shear rate.

TABLE 9
Viscosity of Aged pH Dependent Shell Compositions Aged

| | Aging Time | Viscosity | % Viscosity Reduction |
|---|---|---|---|
| | (hours) | (cPs) | from aging time of 0 hours |
| Non- | 0 | 140,000 | N/A |
| amidated | 24 hours at 60° C. | 90,000 | About 36% |
| pectin | 48 hours at 60° C. | 90,000 | About 36% |
| | 72 hours at 60° C. | 75,000 | About 46% |
| | 96 hours at 60° C. | 75,000 | About 46% |
| Amidated | 0 | 105,000 | N/A |
| Pectin | 24 hours at 60° C. | 70,000 | About 33% |
| | 48 hours at 60° C. | 55,000 | About 48% |
| | 72 hours at 60° C. | 35,000 | About 67% |
| | 96 hours at 60° C. | 40,000 | About 62% |

As can be seen from Table 9, the viscosity of non-amidated pectin decreases by a smaller percentage as compared to the viscosity of amidated pectin, after 48 hours of aging at 60° C., 72 hours of aging at 60° C., and 96 hours of aging at 60° C.

The decrease in viscosity is believed to be caused by the thermal degradation of the molecular chain lengths of pectin and gelatin. Despite this viscosity reduction, the gel masses of the pH dependent shell compositions maintain the viscosity suitable for manufacturability and machinability even after holding the composition in heat at 60° C. for 4 days. Furthermore, softgel capsules manufactured with the aged gel still have satisfactory pH dependent delayed release properties.

### Example 7—Chemical Stability of the Pectin Softgel Capsule

Table 10 below depicts the chemical stability of fish oil encapsulated in a pectin pH dependent shell composition, according to embodiments described herein, after storage for 6 months at ambient conditions and at 40° C. and 75% relative humidity (RH). Acceptable capsules should have EPA TG≥160 mg/g, DHA TG≥100 mg/g, peroxide≤5 meq O₂/kg, p-Anisidine≤20, a dissolution time of more than 120 minutes in 0.1N HCl (pH 1.2), and a dissolution time of up to 45 minutes in buffer medium (pH 6.8 phosphate buffer). The values for these parameters are summarized in Table 10 for the control (fish oil raw material), the delayed release softgel pectin capsule stored at ambient conditions for 6 months, and the delayed release pectin softgel capsule stored at 40° C. and 75% RH for 6 months.

TABLE 10
Chemical Stability of Modified release softgel capsules

| | EPA | DHA | Peroxide | | Dissolution | |
|---|---|---|---|---|---|---|
| | TG | TG | (≤5 | p- | 0.1N | pH6.8 |
| Sample | (≥160 | (≥100 | meq | Anisidine | HCl | Phosphate |
| Lot 10 | mg/g) | mg/g) | O₂/kg) | (≤20) | pH 1.2 | Buffer |
| Fish | 172 | 124 | 0.9 | 11.0 | N/A | N/A |
| Oil Raw | | | | | | |
| Material | | | | | | |
| At T = 6 | 174 | 123 | 2.4 | 12.9 | Pass | 15 |
| months | | | | | (Intact | minutes |
| Ambient | | | | | for 120 | |
| | | | | | minutes) | |
| At T = | 174 | 123 | 2.5 | 14.8 | Pass | 25 |
| 6 months | | | | | (Intact | minutes |
| 40° C./ | | | | | for 120 | |
| 75% RH | | | | | minutes) | |

The accelerated stability data (summarized in Table 10) demonstrates that the pH resistant pectin shell composition, according to embodiments, protected the fill composition (e.g., fish oil constituents) from oxidation, as evident from the insignificant/substantial similarity in the peroxide and p-Anisidine values and EPA and DHA assays after 6 months (at ambient conditions as well as at stressed conditions of 40° C. and 75% RH) as compared to the raw material.

### Example 8—Valproic Acid Pectin Softgel Capsule

Table 11A below depicts the stability of the dissolution profile of valproic acid encapsulated in a pectin pH dependent shell composition (the gel formula of the dry shell composition is summarized in Table 11B), according to embodiments described herein, at T=0, after storage for 3 months (T=3 months) at 40° C. and 75% relative humidity (RH), and after storage for 6 months (T=6 months) and at 40° C. and 75% RH. As evidenced in Table 11A, the dissolution profile of the pH dependent shell composition, after storage for 3 months at 40° C. and 75% RH and after storage for 6 months at 40° C. and 75% RH, remains substantially similar to the dissolution profile at T=0.

TABLE 11A
Dissolution of Valproic Acid Encapsulated In A Pectin pH Dependent
Shell Composition

| | | | | T = 3 Months | | T = 6 Months | |
|---|---|---|---|---|---|---|---|
| | | T = 0 | | 40° C./75% RH | | 40° C./75% RH | |
| | | | Buffer | | Buffer | | Buffer |
| | | | Stage | | Stage | | Stage |
| | | Acid Stage | (pH 6.8 | Acid Stage | (pH 6.8 | Acid Stage | (pH 6.8 |
| Lot | Fill | (0.1N HCl, | phosphate | (0.1N HCl, | phosphate | (0.1N HCl, | phosphate |
| No. | composition | pH 1.2) | buffer) | pH 1.2) | buffer) | pH 1.2) | buffer) |
| Lot | Valproic | Intact | Pass | Intact | Pass | Intact | Pass |
| 15 | Acid | (120 | (9 | (120 | (12 | (120 | (11 |
| | | minutes) | min) | minutes) | min) | minutes) | min) |

TABLE 11B
Gel Mass Formulations in wt. % in Dry Capsule Shell

| Ingredient | Lot 15 |
|---|---|
| Amidated pectin | 6.5-8.0 |
| Dextrose | None |
| Glycerin | 20-45 |
| Gelatin | 42-56 |
| Water | 8-15 |
| Total | 100 |

### Example 9—Physical Attributes of Pectin Softgel Capsule

Modified release softgel capsules having the pH dependent shell composition described herein are robust as evidenced based on the physical attributes summarized in Table 12 below.

TABLE 12
Physical Attributes of Modified release softgel capsules

| Parameters | Typical Specifications |
|---|---|
| Shell Moisture (%) | 6-15 |
| Hardness (Newtons) | 7-14 |
| Equilibrium Relative Humidity (%) | 30-45 |
| Burst Strength (kg) | 60-120 |

The shell moisture was determined by loss on drying method. A pH dependent capsule shell composition sample of 1 to 2 grams were placed into a 105° C. oven for 17 hours. The initial weight of the sample was recorded. After drying the sample in the oven at 105° C. for 17 hours, the final weight of the sample was recorded. The percentage of weight loss, calculated in accordance with the below equation, was defined as the shell moisture:

$$\% weight lost = \frac{( initial weight ) - ( final weight )}{( initial weight )} \cdot 100 \%$$

The capsule hardness was determined using a hardness tester. The force required to cause a 2.0 mm deformation of the capsule in Newton was defined as the capsule hardness.

Equilibrium Relative Humidity (%) was defined as the humidity condition at which the capsule maintained a constant total weight. It was determined using environmental chambers maintained at constant humidity using saturated salt solutions.

Burst strength was determined using a texture analyzer. The texture analyzer compressed the capsule until the capsule burst. The force, in kilograms, required to make the capsule burst was defined as burst strength.

### Example 10—Exemplary Composition of a Pectin and Gellan Gum Modified Release Softgel Capsule

Modified release softgel capsule that includes a combination of pectin and gellan gum was prepared. The formulation based on dry shell composition is summarized in Table 13 below.

TABLE 13
Gel Mass Formulations in wt. % in Dry Capsule Shell

| Ingredient | Lot 15 |
|---|---|
| Pectin | 7.0-10.5 |
| Dextrose | 0.02-0.5 |
| Glycerin | 15-25 |
| Gelatin | 35-50 |
| Sorbitol Solution | 25-32 |
| Gellan Gum | 0.1-2.0 |
| Water | 6-15 |
| Total | 100 |

Examples 1-10 exemplify a first level of controlled release in the dual controlled release softgel capsules described herein. The first level being the pH dependent shell composition. Examples 11-12 exemplify a dual level of controlled release, where the first level can be attributed to the pH dependent shell composition and the second level can be attributed to the controlled release fill composition.

### Example 11—Exemplary Composition of a Pectin and Gellan Gum Modified Release Softgel Capsule with a Controlled Release Fill Composition

Dual controlled release softgel capsule that includes a pH dependent shell composition, described in Table 14, and a controlled release fill composition, described in Table 15, was prepared.

TABLE 14
Gel Mass Formulations in wt. % in Dry Capsule Shell

| Ingredient | wt. % range |
|---|---|
| Gelatin | 28-50 |
| Sorbitol Solution | 15-35 |
| Pectin | 2.5-7.0 |
| Gellan gum | 0.1-2.0 |
| Dextrose | 0.005-0.5 |
| Purified Water | 25-45 |
| Total | 100.0 |

TABLE 15
Controlled Release Fill Compositions Containing Polyox ™

| Ingredient | Fill-1 (%) | Fill-2 (%) | Fill-3 (%) |
|---|---|---|---|
| Water | 1.0-4.0 | 1.5-5.0 | 2.0-6.0 |
| Potassium hydroxide | 1.0-3.0 | 1.5-4.0 | 2.0-5.0 |
| Polyethylene glycol 600 | 15.0-28.0 | 20.0-42.0 | 22.0-45.0 |
| Ibuprofen | 12.0-18.0 | 19.0-25.0 | 24.0-32.0 |
| Polyethylene glycol 400 | 40.0-45.0 | 20.0-32.0 | 5.0-15.0 |
| Polyethylene oxide | 8.0-15.0 | 16.0-20.0 | 22.0-28.0 |
| Total | 100.0 | 100.0 | 100.0 |

The grade of Polyethylene Oxide polymer used in the controlled release fill compositions summarized in Table 15 was POLYOX™ WSR 301. POLYOX™ WSR 301 is a water-soluble polymer. It is based on a long chain nonionic polyethylene oxide polymer. POLYOX™ WSR 301 has a high molecular weight of 4,000,000 Dalton with a viscosity of 1650-5500 cPs.

Modified release softgel capsules were prepared by encapsulating each of the controlled release fill composition from Table 15 in the pH dependent shell composition from Table 14. After encapsulation, the modified release softgel capsules were dried. After the capsules were dried, the capsules were annealed at 60° C. for between 1 to 3 hours. After annealing, the capsules were allowed to cool to ambient conditions. Annealing facilitated melting of the Polyethylene Oxide polymer to form a solid matrix inside the capsule.

Dissolution tests were performed on the resulting capsules. A USP Apparatus II with paddles at 50 rpm was used. During the first 120 minutes, 0.1N HCl was used as dissolution medium. Then phosphate buffer was added to adjust the pH to 6.8. The drug release profiles were monitored using fiber optic probes for up to 24 hours. FIG. 2 shows the release profile of Ibuprofen from the capsules.

As seen in FIG. 2, during the first two hours in 0.1N HCl medium, no Ibuprofen was released, indicating robust enteric property of the pH dependent shell composition. Of note is that release of up to 10% is allowed per the Pharmacopeia in acidic media. When the pH was adjusted to pH 6.8, Ibuprofen was gradually released from the Polyethylene Oxide polymer matrix (i.e., from the controlled release fill composition).

The amount of Polyethylene Oxide polymer had an effect on the rates of Ibuprofen release. The higher the amount of Polyethylene Oxide polymer, the slower the Ibuprofen release was. Formulation Fill-3 containing 24 wt. % Polyethylene Oxide polymer achieved a zero order release profile over a duration of 24 hours. Formulations Fill-1 and Fill-2 containing 12 wt. % and 18 wt. % Polyethylene Oxide polymer, respectively, achieved a 12 hour release profiles.

This data showed that the drug release profile can be modulated by altering the fill composition.

### Example 12—Exemplary Composition of a Pectin Modified Release Softgel Capsule with a Controlled Release Fill Composition

Dual controlled release softgel capsule that includes a pH dependent shell composition, described in Table 16, and a controlled release fill composition, described in Table 17 (diphenhydramine) and in Table 18 (Acetaminophen), were prepared. The controlled release fill compositions were encapsulated in a pH dependent shell composition and thereafter the capsules were dried.

TABLE 16
Gel Mass Formulations in wt. % in Dry Capsule Shell

| Ingredient | %, w/w | Function |
|---|---|---|
| Amidated pectin | 3.5-6.5 | Enteric polymer |
| Glycerin | 12.0-21.0 | Plasticizer |
| Dextrose | 0.005-0.4 | Enhancer |
| Gelatin | 28.0-45.0 | Film former |
| Water | 33.0-51.0 | Solvent |
| Total | 100.0 | |

TABLE 17
Controlled Release Fill Composition Containing Diphenhydramine

| Ingredient | Fill-4 (%) |
|---|---|
| Polyethylene glycol 400 | 40.0-55.0 |
| Polyethylene Oxide (POLYOX ™ WSR 301) | 15.0-30.0 |
| Water | 3.0-7.0 |
| Xanthan Gum | 5.0-12.0 |
| Diphenhydramine | 4.0-10.0 |
| Total | 100.0 |

TABLE 18
Controlled Release Fill Composition Containing Acetaminophen

| Ingredient | Fill-5 (%) |
|---|---|
| Polyethylene glycol 400 | 35.0-55.0 |
| Polyethylene Oxide (POLYOX ™ WSR 301) | 10.0-35.0 |
| Hydroxypropyl methylcellulose | 1.0-5.5 |
| Acetaminophen | 25.0-42.0 |
| Total | 100.0 |

Each Diphenhydramine capsule contained 50 mg Diphenhydramine. Each Acetaminophen capsule contained 325 mg Acetaminophen.

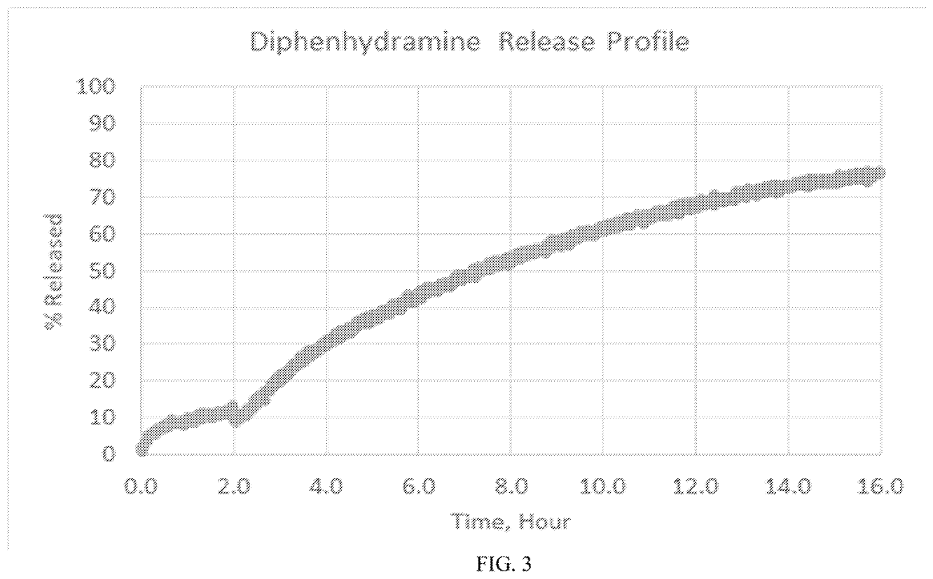

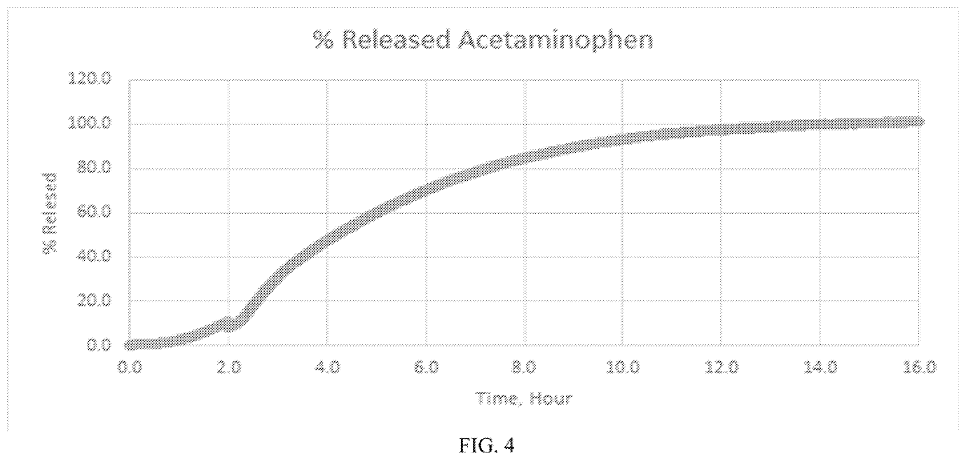

The filled capsules were then placed into a 65° C. oven for 90 minutes to anneal the capsules. After annealing the capsules were subjected to two-stage dissolution tests. The paddle speed was set at 50 RPM. During the first 120 minutes, 0.1N HCl was used as dissolution medium. Then phosphate buffer was added to adjust the pH to 6.8. The drug release profiles were monitored using fiber optic probes for up to 12 hours. FIGS. 3 and 4 show the releases of Diphenhydramine and Acetaminophen from the capsules, respectively.

In summary, the combination of pectin in the shell compositions and Polyox™ polymer in the fill compositions resulted in a new drug delivery platform that has the potential to be used for colonic drug delivery and other drug delivery application.

### Examples 13-18 Dissolution Profiles of Fill Compositions

A 2×3 full factorial design of experiment with duplicates was utilized for the design of the six (6) fill compositions used in Samples 1-12 as set forth in Table 19 below. Each of the compositions was prepared twice to enable assessment of the composition variability. Diphenhydramine HCl was used as a model drug for the active pharmaceutical ingredient in the fill compositions. “PEG 400” is an abbreviation for polyethylene glycol having a number average molecular weight of 400, “PEO” is an abbreviation for polyethylene oxide, “M” stands for “million”, “HCl” is an abbreviation for hydrogen chloride and “Mn” is an abbreviation for number average molecular weight. All PEO's used in Samples 1-12 were non-ionic and water soluble and were Polyox™ products obtainable from DuPont Pharma Solutions.

TABLE 19
Fill Compositions

| | PEG | PEO | | | Diphen- |
|---|---|---|---|---|---|
| | 400 | Polyox ™ | PEO | Water | hydramine |
| Samples | (g) | Grade | (g) | (g) | HCl (g) |
| 1 | 14.0 | Mn 5M Da | 4.0 | 2.0 | 2.0 |
| 2 | 14.0 | Mn 0.9M Da | 4.0 | 2.0 | 2.0 |
| 3 | 14.0 | Mn 0.1M Da | 4.0 | 2.0 | 2.0 |
| 4 | 14.0 | Mn 5M Da | 4.0 | 2.0 | 2.0 |
| 5 | 10.0 | Mn 5M Da | 8.0 | 2.0 | 2.0 |
| 6 | 10.0 | Mn 0.1M Da | 8.0 | 2.0 | 2.0 |
| 7 | 10.0 | Mn 5M Da | 8.0 | 2.0 | 2.0 |
| 8 | 14.0 | Mn 0.9M Da | 4.0 | 2.0 | 2.0 |
| 9 | 10.0 | Mn 0.1M Da | 8.0 | 2.0 | 2.0 |
| 10 | 10.0 | Mn 0.9M Da | 8.0 | 2.0 | 2.0 |
| 11 | 10.0 | Mn 0.9M Da | 8.0 | 2.0 | 2.0 |
| 12 | 14.0 | Mn 0.1M Da | 4.0 | 2.0 | 2.0 |

The diphenhydramine capsules of Samples 1-12 using the fill compositions set forth in Table 19 were prepared as follows. First, the fill compositions were made by solubilizing the diphenhydramine HCl (DHP) in 2 ml of water and mixing the PEG 400 with the PEO to form two components. The aqueous DPH solution was then added to the PEG/PEO mixture. Each Size 0 capsule was filled with 0.55 g of the fill composition to provide a dose of 50 mg diphenhydramine per capsule. The capsules were then annealed at ° C. for one (1) hour in an oven.

The dissolution studies were carried out using the prefilled Size 0 gelatin hardshell capsules containing fill compositions by fiberoptic dissolution using USP Apparatus II with paddle speeds of 50 rpm and 100 rpm, at 37° C. in 500 ml water as the dissolution medium. The fill compositions used for the dissolution studies are shown in Table 20.

TABLE 20
Fill Compositions Used for Dissolution Studies

| | PEG | PEO | | Diphen- | |
|---|---|---|---|---|---|
| | 400 | Polyox ™ | PEO | hydramine | Water |
| Formulation | (g) | Grade | (g) | (g) | (g) |
| 1 (Exam- | 10.0 | Mn 5M Da | 8.0 | 2.0 | 2.0 |
| ple 13) | | | | | |
| 2 (Exam- | 14.0 | Mn 5M Da | 4.0 | 2.0 | 2.0 |
| ple 14) | | | | | |
| 3 (Exam- | 10.0 | Mn 0.9M Da | 8.0 | 2.0 | 2.0 |
| ple 15) | | | | | |
| 4 (Exam- | 14.0 | Mn 0.9M Da | 4.0 | 2.0 | 2.0 |
| ple 16) | | | | | |
| 5 (Exam- | 10.0 | Mn 0.1M Da | 8.0 | 2.0 | 2.0 |
| ple 17) | | | | | |
| 6 (Exam- | 14.0 | Mn 0.1M Da | 4.0 | 2.0 | 2.0 |
| ple 18) | | | | | |

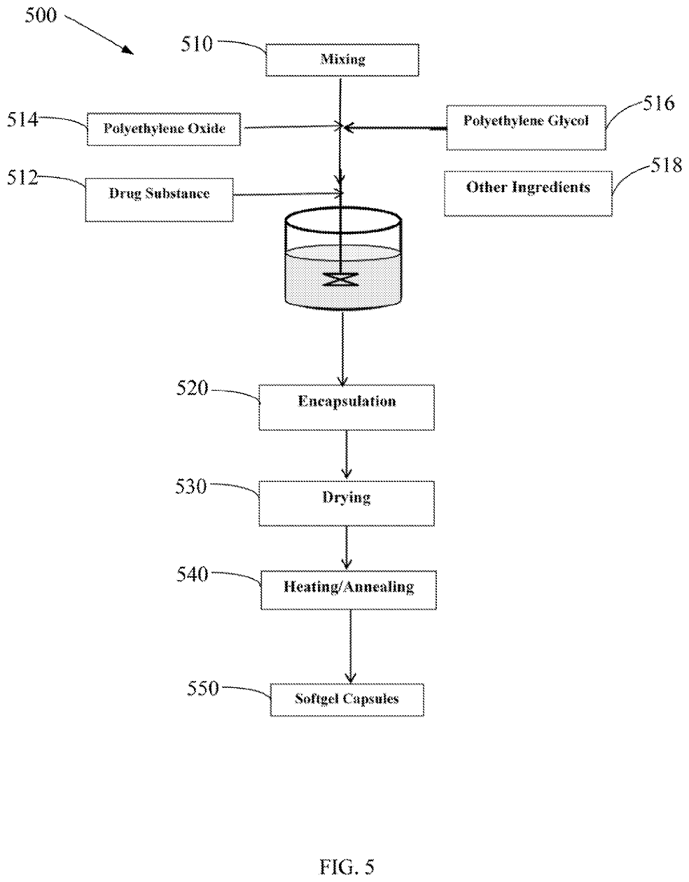

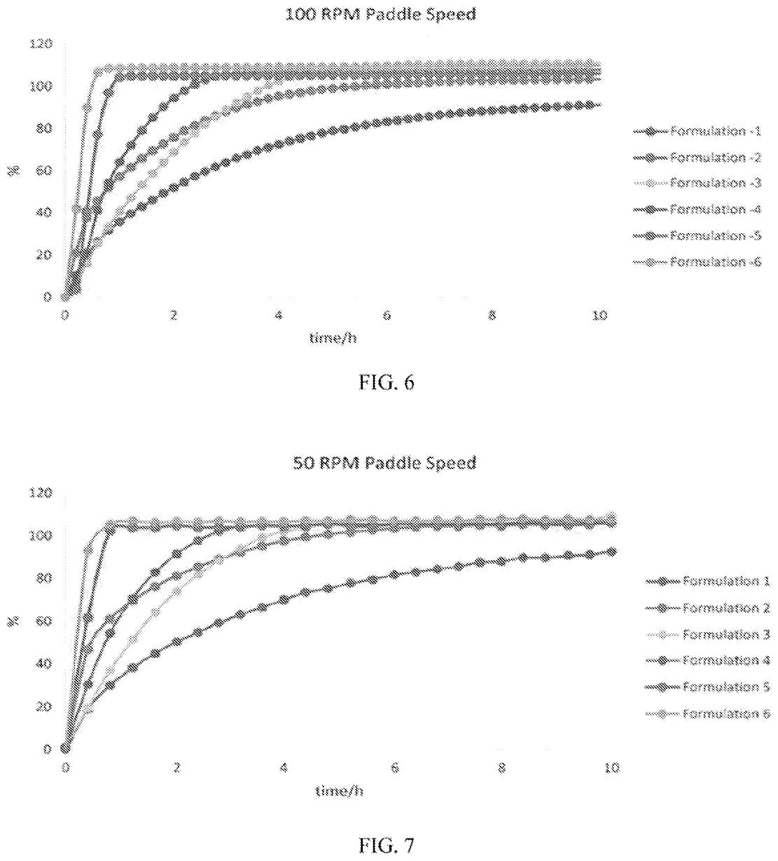

The dissolution profiles for the six (6) fill compositions listed in Table 20 at 100 RPM paddle speed are shown in FIG. 6. The dissolution profiles for the six (6) fill compositions listed in Table 20 at 50 RPM paddle speed are shown in FIG. 7.

The dissolution profiles were similar at 50 RPM and 100 RPM paddle speeds for each fill composition, indicating that the drug release mechanism was mainly by diffusion. The dissolution results show that higher molecular weight PEO and higher PEO concentration each resulted in a slower drug release. Fill compositions 5 and 6 prepared from 0.1M PEO had immediate release profiles while all the other fill compositions exhibited variable drug release rates as shown in FIGS. 6-7.

The Minitab 16 software package was used to analyze the collected dissolution data set. The time for the drug release to reach 90% was used as the dependent variable. The effect of PEO content and PEO molecular weight on the dependent variable was analyzed using the General Linear Model module in the Minitab 16 software package. The results are summarized in the following tables 21-22\24.

TABLE 21
General Linear Model: time to release
90% DHP v. PEO %, PEO Mn (MDa)

| Factor | Type | Levels | Values | | |
|---|---|---|---|---|---|
| PEO % | Fixed | 2 | 18.182 | 36.364 | |
| PEO Mn | Fixed | 3 | 0.1 | 0.9 | 5.0 |
| (MDa) | | | | | |

TABLE 22
Analysis of Variance for time 90% (h), using Adjusted SS for Tests

| Source | DF | Seq SS | Adj SS | Adj MS | F | P |
|---|---|---|---|---|---|---|
| PEO % | 1 | 29.482 | 29.482 | 29.482 | 22.14 | 0.000 |
| PEO Mn (MDa) | 2 | 116.323 | 116.323 | 58.162 | 43.68 | 0.000 |
| PEO % *PEO Mn | 2 | 22.703 | 22.703 | 11.352 | 8.52 | 0.002 |
| (MDa) | | | | | | |
| Error | 18 | 23.970 | 23.970 | 1.332 | | |
| Total | 23 | 192.478 | | | | |

| | | |
|---|---|---|
| S = 1.15398 | R-Sq = 87.55% | R-Sq(adj) = 84.09% |

TABLE 23
Grouping Information Using Tukey Method and 95.0% Confidence

| PEO % | N | Mean | Grouping |
|---|---|---|---|
| 36.364 | 12 | 4.1500 | A |
| 18.182 | 12 | 1.9333 | B |

Means that do not share a letter are significantly different.

TABLE 24
Grouping Information Using Tukey Method and 95.0% Confidence

| PEO Mn (MDa) | N | Mean | Grouping |
|---|---|---|---|
| 5.0 | 8 | 5.9500 | A |
| 0.9 | 8 | 2.5500 | B |
| 0.1 | 8 | 0.6250 | C |

Means that do not share a letter grouping are significantly different.

In the foregoing tables, the following abbreviations were employed:
DF—Degrees of Freedom
Seq SS—Sequential sums of squares which are measures of variation for different components of the model.
Adj SS—Adjusted sum of squares for a term is the increase in the regression sum of squares compared to a model with only the other terms
Adj MS—Adjusted mean squares measure how much variation a term or a model explains
F—F-value is the test statistic used to determine whether the model is missing higher-order terms that include the predictors in the current model.
P—Probability. P<0.05 indicates that the result is significant; otherwise, it is not significant.
N—Number of data points

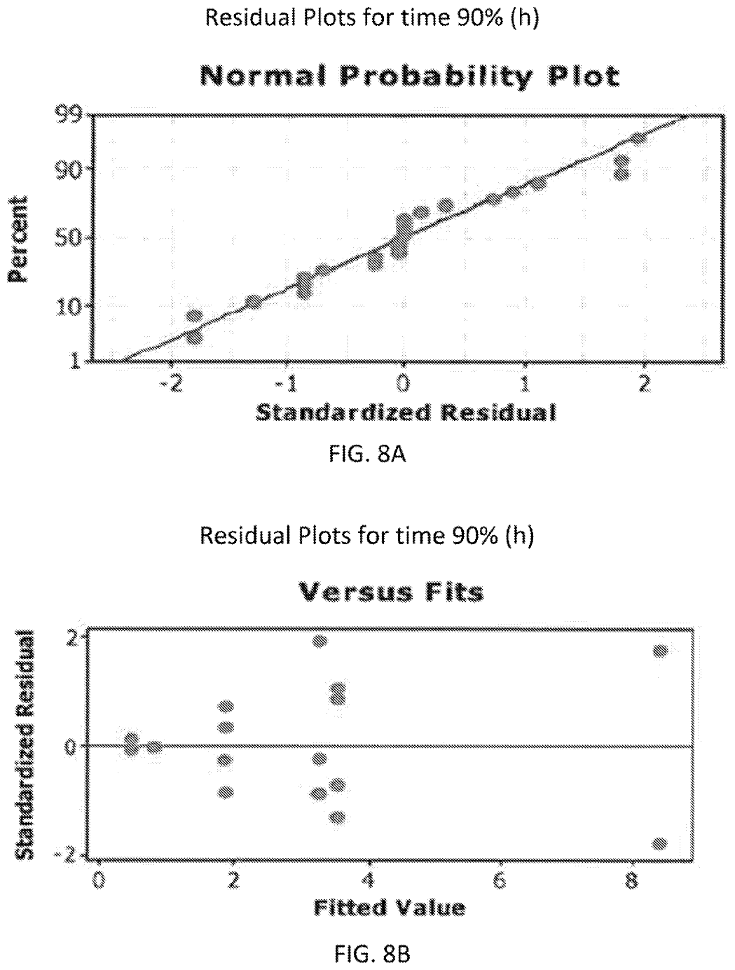

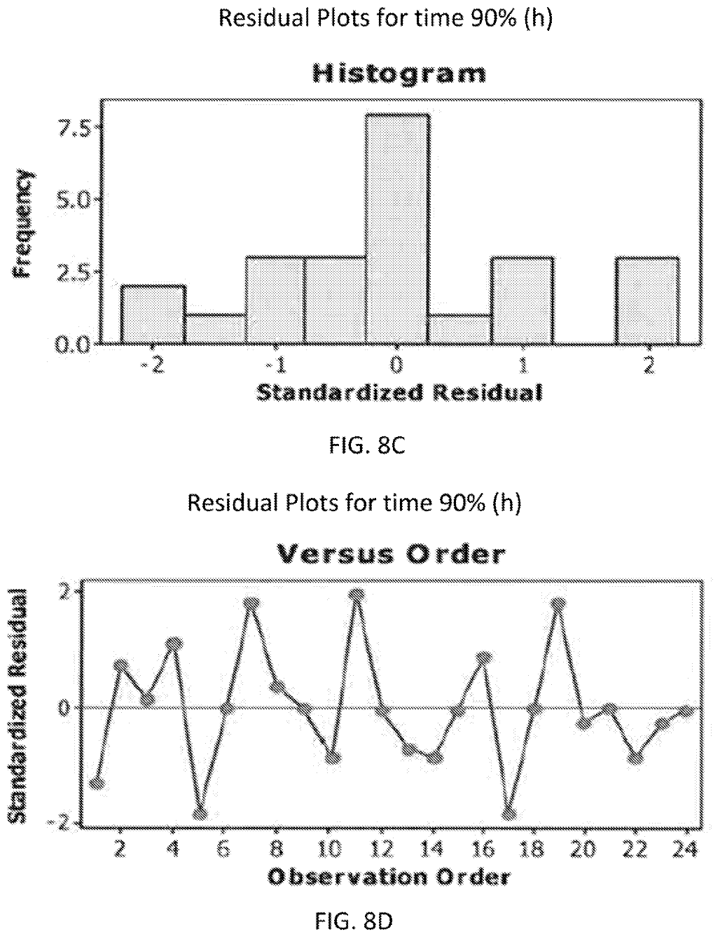

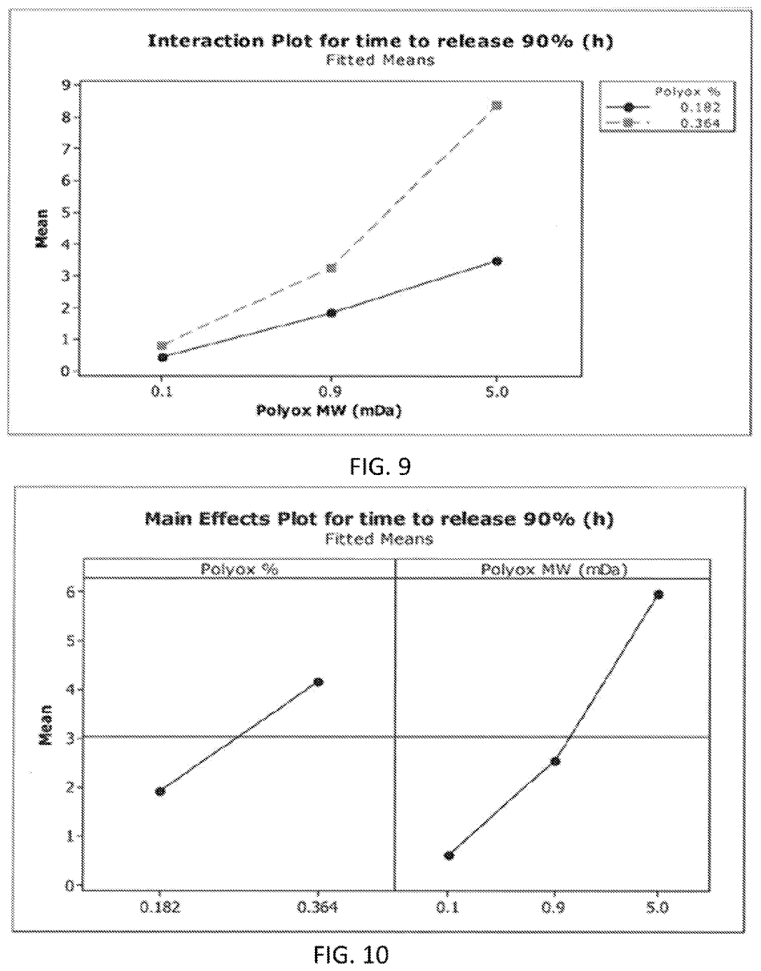

FIGS. 8A-8D shows the residual plots for time 90% (hours). FIG. 8A is a normal probability plot, FIG. 8B is a versus fits, FIG. 8C is a histogram, and FIG. 8D is a versus order. FIG. 9 shows the interaction plot for time to release 90% (hours). FIG. 10 is a graph showing the main effect plot for time to release 90% (hours).

Based on these statistical analyses, there is an interaction between the time to release and the PEO molecular weight and concentration. The higher the PEO molecular weight, and the higher the PEO concentration, the slower the API release.

### Example 19—PEO Polymer, High Mn Polyethylene Glycol, and HPMC Polymer Immediate Release Compositions

Immediate release compositions based on PEO resins, high molecular weight polyethylene glycol (1000-5000 daltons) and low viscosity hydroxypropyl methylcellulose (HPMC) were developed for potential applications in abuse deterrent softgel capsules. The three (3) formulations shown in Table 25 below were prepared. Formulation 13 contained PEO with a number average molecular weight of 100,000 Da and PEG 3350. Formulation 14 contained PEO and HPMC. Formulation 15 contained PEO, PEG 3350, and HPMC.

TABLE 25
Formulations Containing PEG 3350 and HPMC

| | Formulation | Wt. | Formulation | Wt. | Formula | Wt. |
|---|---|---|---|---|---|---|
| | 13 (g) | % | 14 (g) | % | 15 (g) | % |
| PEO | 6.0 | 30.0 | 6.0 | 30.0 | 4.0 | 20.0 |
| (Mn = 100,000 | | | | | | |
| Da) | | | | | | |
| PEG 400 | 10 | 50.0 | 10 | 50.0 | 10.0 | 50.0 |
| PEG 3350 | 1.0 | 5.0 | — | — | 2.0 | 10.0 |
| HPMC | — | — | 1.0 | 5 | 1.0 | 5.0 |
| METHOCEL ™ | | | | | | |
| VLV | | | | | | |
| Water | 2.0 | 10.0 | 2.0 | 10.0 | 2.0 | 10.0 |
| Diphenhydramine | 1.0 | 5.0 | 1.0 | 5.0 | 1.0 | 5.0 |
| Total (g) | 20.0 | 100.0 | 20.0 | 100.0 | 20.0 | 100.0 |

Size 0 diphenhydramine (DPH) capsules were prepared by mixing PEG 400 with PEO and PEG 3350 and/or HPMC. The DPH was solubilized in water and the DPH solution was added to the PEG/PEO mixture, or the HPMC/PEO mixture, or the PEO/PEO/HPMC mixture. Each capsule was filled with 0.5 g of the fill mixture (25 mg diphenhydramine per capsule). Finally, the capsules were annealed at 60° C. for one (1) hour in an oven.

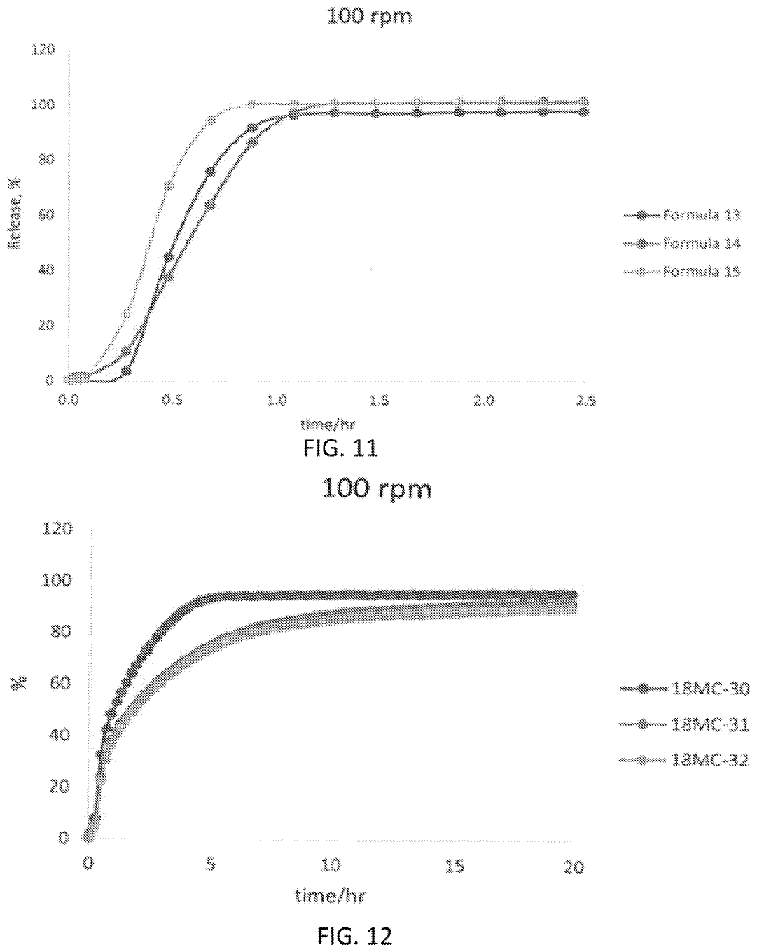

For the dissolution study, fiberoptic dissolution was carried out using USP Apparatus II with paddle speeds of 100 RPM at 37° C. in 500 ml water as the dissolution medium. The dissolution profiles for Formulations 13-15 are shown in FIG. 11.

Formulations 13-15 were shown to be immediate release dosage forms. Diphenhydramine release reached 100% from these formulations in approximately one (1) hour. Formulation 15 had the fastest drug release rate among the three formulations. Not to be bound by theory, but this is believed to have been due to the higher amount of PEG 3350 in Formulation 15.

### Examples 20-22 Controlled Release PEO Softgel Capsules

Three batches of softgel capsules containing fill compositions made from PEO resins with various number average molecular weights (900,000 Da, 5,000,000 Da and 7,000,000 Da) were manufactured using a softgel capsule encapsulation machine. The fill compositions used for batch manufacturing are shown in Tables 26-28 below.

TABLE 26
Fill Formula for Example 20 (18MC-30)

| mg per capsule | Item Description |
|---|---|
| 25.0 | Diphenhydramine HCl, USP |
| 300.0 | Polyethylene Glycol 400, NF |
| 175.0 | Polyethylene oxide - Mn 900,000 Da |
| | (Polyox ™ WSR 1105) |
| Total | |
| 500.0 | |

TABLE 27
Fill Formula for Example 21 (18MC-31)

| mg per capsule | Item Description |
|---|---|
| 25.0 | Diphenhydramine HCl, USP |
| 300.0 | Polyethylene Glycol 400, NF |
| 175.0 | Polyethylene oxide - Mn 5,000,000 Da |
| | (Polyox ™ WSR Coagulant) |
| Total | |
| 500.0 | |

TABLE 28
Fill Formula for Example 22 (18MC-32)

| mg per capsule | Item Description |
|---|---|
| 25.0 | Diphenhydramine HCl, USP |
| 300.0 | Polyethylene Glycol 400, NF |
| 175.0 | Polyethylene oxide - Mn 7,000,000 Da |
| | (Polyox ™ WSR-303) |
| Total | |
| 500.0 | |

After encapsulation, the softgel capsules were sealed in aluminum bags for five (5) days to allow moisture migration from the wet capsule shell into the fill. This moisture migration was utilized to solubilize the PEO in the fill composition, and to form gels to provide sustained release profiles. After five (5) days, the fill moisture of each of the capsules was tested and the results are shown in Table 29 below.

TABLE 29
Softgel Capsule Fill Moisture

| | Fill Moisture, wt. % water | | |
|---|---|---|---|
| Example | Sample 1 | Sample 2 | Average |
| 20 (18MC-30) | 19.3 | 16.2 | 17.3 |
| 21 (18MC-31) | 17.1 | 16.2 | 16.7 |
| 22 (18MC-32) | 17.2 | 17.3 | 17.3 |

Although the fill moistures were high enough, the results showed that the PEO resin particles inside the softgel capsules did not fully solubilize. Without being bound by theory, it appears that the PEG 400 bound the fill moisture making it unavailable to fully solubilize the PEO resin particles. Thus, the softgel capsules were annealed at 60° C. for one (1) hour in an oven to melt and solubilize the PEO resin particles. The annealed softgel capsules were then subjected to dissolution tests.

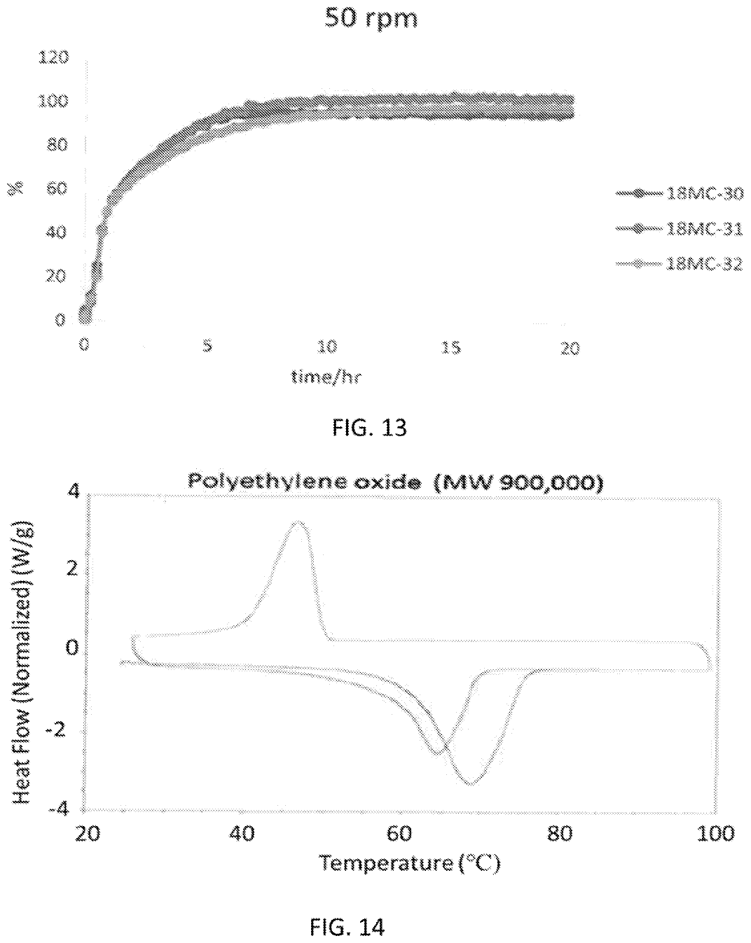

Fiberoptic dissolution using USP Apparatus II with paddle speeds of 50 RPM and 100 RPM at 37° C. in 500 ml water dissolution medium was employed to evaluate the drug release rate in vitro. The comparative dissolution results for capsules prepared with three (3) PEO resins of varying number average molecular weights are shown in FIGS. 12-13.

At the 100 RPM paddle speed, capsules containing PEO with a 900,000 Da number average molecular weight showed a faster drug release rate as compared to capsules prepared with PEO with either a 5,000,000 or a 7,000,000 Da number average molecular weight. The capsules prepared with PEO having the 5,000,000 and 7,000,000 Da number average molecular weights showed similar drug release rates. At 50 RPM, the dissolution profiles were similar for all three of the capsules of Examples 20-22.

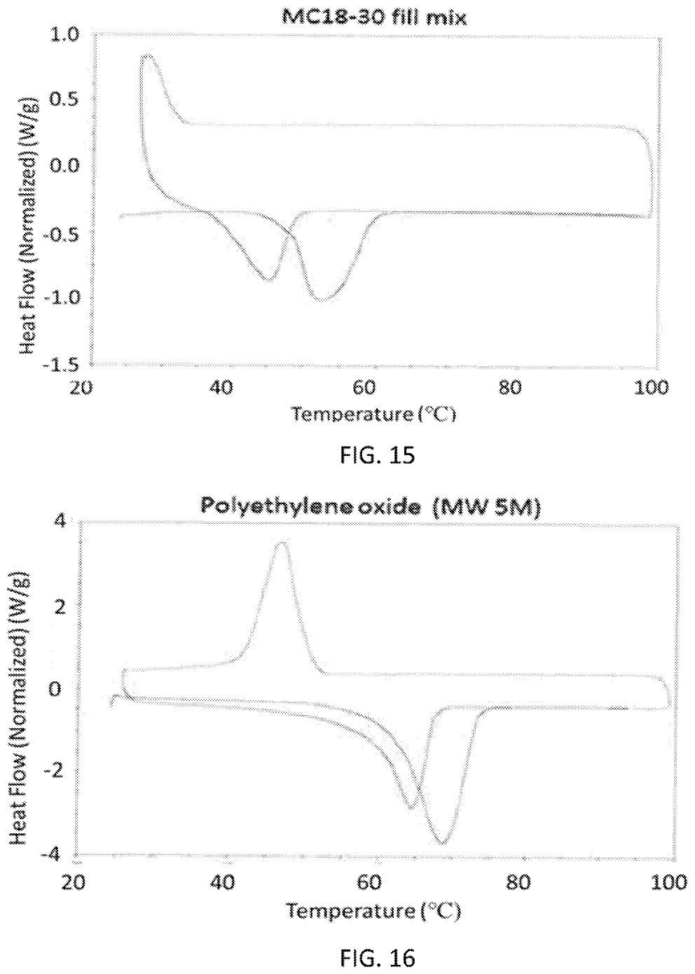

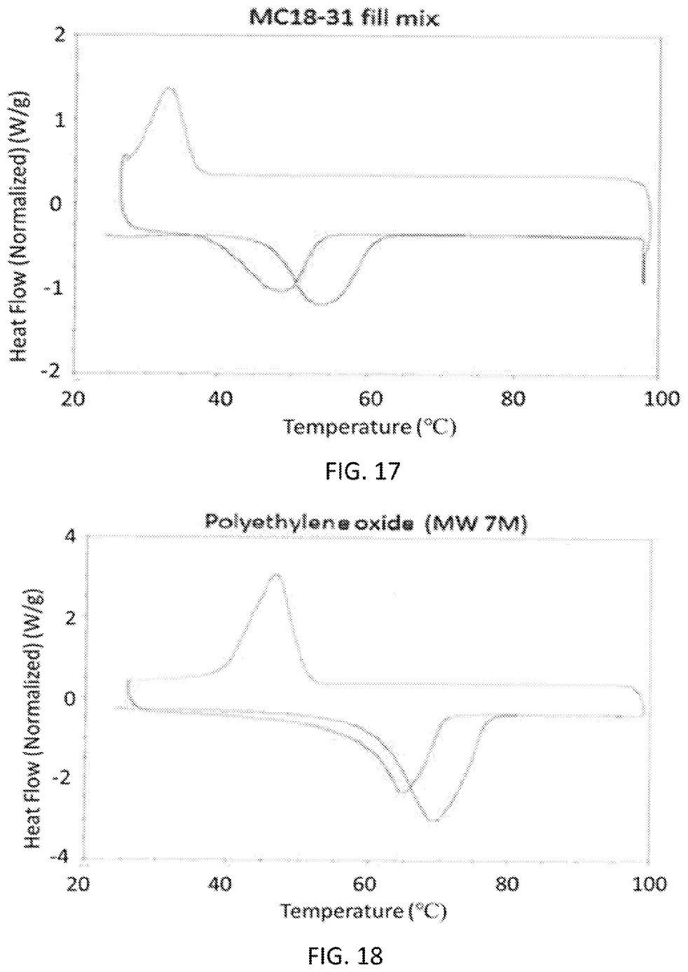

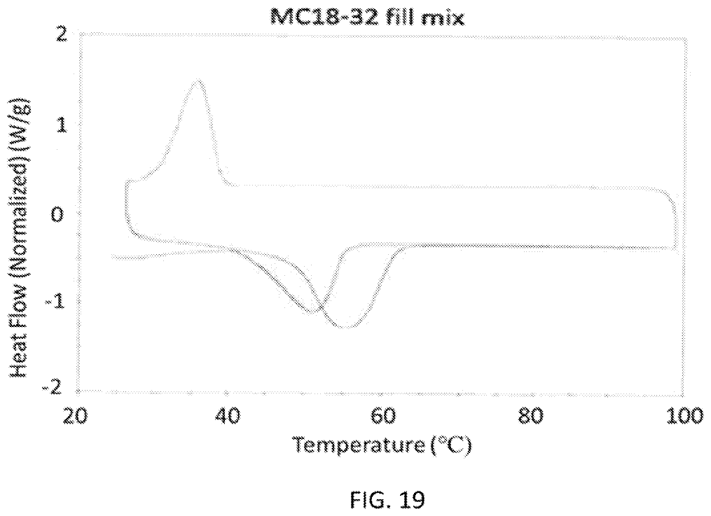

Differential Scanning Colorimetry (DSC) analyses were performed on the PEO resins and the fill compositions used for softgel encapsulation as shown in FIGS. 14-19. The blue curves represent the initial heating at 10° C. per minute. The green curves represent cooling at 10° C. per minute. The red curves represent a second heating at 10° C. per minute. All three PEO resins had melting temperatures below 60° C. upon the initial heating cycle. Not to be bound by theory, this lowered melting temperature of the fill compositions was believed to be due to a plasticizing effect of PEG 400 on the PEO resins. The DSC analyses can be employed to select the proper processing temperature and annealing temperature for the specific fill composition.

Controlled release softgel fill compositions based on polyethylene oxide resins were developed per design of experiment. The effects of PEO concentration and molecular weight on drug release rate were studied. The drug release rate was significantly affected by both the molecular weight of the PEO and the PEO polymer concentration. The higher the PEO molecular weight or the PEO polymer concentration, the slower the drug release rate. The dissolution profiles were similar for the same composition with either a 50 rpm or a 100 rpm paddle speed, indicating that the drug release mechanism was mainly due to diffusion through the polymer matrix.

Compositions containing low molecular weight PEO, PEG 3350 and low viscosity HPMC were also developed for immediate release softgel capsules. These compositions showed immediate release profiles when subjected to dissolution studies.

Three batches of softgel capsules containing various Mn PEO resins were manufactured. The softgel capsules were subjected to dissolution tests. All three batches of softgel capsules show extended release profiles. DSC analyses were performed on the PEO resins and the three compositions. PEG 400 in the composition appears to act as a plasticizing agent to PEO resins, resulting in lower melting temperatures (<60° C.) for the PEO resins, which is beneficial for product manufacture.

### Example 23—Viscosity Adjustment of Fill Compositions Using Polyethylene Oxide

Three compositions containing only polyethylene oxide (Polyox™) and polyethylene glycol 400 were made to demonstrate how the viscosity of the fill compositions can be controlled by varying the amounts of polyethylene oxide and polyethylene glycol in the fill compositions. The fill compositions and their viscosities are shown in Table 30 below.

TABLE 30
Viscosity Adjustment

| PEO (wt. %) | PEG 400 wt. % | Viscosity (cP) |
|---|---|---|
| 10 | 90 | 229 |
| 30 | 70 | 2374 |
| 40 | 60 | 18190 |

For simplicity of explanation, the embodiments of the methods of this disclosure are depicted and described as a series of acts. However, acts in accordance with this disclosure can occur in various orders and/or concurrently, and with other acts not presented and described herein. Furthermore, not all illustrated acts may be required to implement the methods in accordance with the disclosed subject matter. In addition, those skilled in the art will understand and appreciate that the methods could alternatively be represented as a series of interrelated states via a state diagram or events.

In the foregoing description, numerous specific details are set forth, such as specific materials, dimensions, processes parameters, etc., to provide a thorough understanding of the present disclosure. The particular features, structures, materials, or characteristics may be combined in any suitable manner in one or more embodiments. The words “example” or “exemplary” are used herein to mean serving as an example, instance, or illustration. Any aspect or design described herein as “example” or “exemplary” is not necessarily to be construed as preferred or advantageous over other aspects or designs. Rather, use of the words “example” or “exemplary” is intended to present concepts in a concrete fashion. As used in this application, the term “or” is intended to mean an inclusive “or” rather than an exclusive “or”. That is, unless specified otherwise, or clear from context, “X includes A or B” is intended to mean any of the natural inclusive permutations. That is, if X includes A; X includes B; or X includes both A and B, then “X includes A or B” is satisfied under any of the foregoing instances. Reference throughout this specification to “an embodiment”, “certain embodiments”, or “one embodiment” means that a particular feature, structure, or characteristic described in connection with the embodiment is included in at least one embodiment. Thus, the appearances of the phrase “an embodiment”, “certain embodiments”, or “one embodiment” in various places throughout this specification are not necessarily all referring to the same embodiment.

The present disclosure has been described with reference to specific exemplary embodiments thereof. The specification and drawings are, accordingly, to be regarded in an illustrative rather than a restrictive sense. Various modifications of the disclosure in addition to those shown and described herein will become apparent to those skilled in the art and are intended to fall within the scope of the appended claims.

1. A modified release softgel capsule comprising:
(a) a controlled release fill composition comprising:
(i) at least one active agent; and
(ii) a controlled release material; and
(b) a pH dependent shell composition encapsulating the fill composition, wherein the pH dependent shell composition comprises gelatin, pectin, and dextrose.

2. The modified release softgel capsule of claim 1, wherein the controlled release material comprises polyethylene oxide, cellulose derivative, a gum, or a combination thereof.

3. The modified release softgel capsule of claim 2, wherein the controlled release material comprises polyethylene oxide having a number average molecular weight of from about 0.05M Dalton to about 15M Dalton.

4. The modified release softgel capsule of claim 2, wherein the controlled release material comprises a cellulose derivative selected from the group consisting of microcrystalline cellulose, sodium carboxymethyl cellulose, methylcellulose, ethyl cellulose, hydroxyethyl cellulose, hydroxypropyl cellulose, hydroxypropyl methylcellulose, and a combination thereof.

5. The modified release softgel capsule of claim 2, wherein the controlled release material comprises a gum selected from the group consisting of gum tragacanth, gum acacia, guar gum, xanthan gum, locust bean gum, and a combination thereof.

6. The modified release softgel capsule of claim 1, wherein the controlled release material is present in the controlled release fill composition in an amount of at least about 8 wt. % and up to about 65 wt. %, based on a total weight of the controlled release fill composition.

7. The modified release softgel capsule of claim 1, wherein the controlled release fill composition further comprises (iii) a hydrophilic carrier having a number average molecular weight of from about 200 Daltons to about 5000 Daltons.

8. The modified release softgel capsule of claim 7, wherein the hydrophilic carrier comprises polyethylene glycol, propylene glycol, water, or a combination thereof.

9. The modified release softgel capsule of claim 7, wherein the hydrophilic carrier is present in the controlled release fill composition in an amount of above 0 wt. % and up to about 70 wt. %, based on a total weight of the controlled release fill composition.

10. The modified release softgel capsule of claim 1, wherein the at least one active agent is present in the controlled release fill composition in an amount of about 5 wt. % to about 60 wt. %, based on the total weight of the controlled release fill composition.

11. The modified release softgel capsule of claim 1, wherein less than about 85% is released after 0.5 hours based on a fiber optic dissolution test using Apparatus II using a paddle speed of 50 rpm in pH 6.8 phosphate buffer, optionally with Pancreatin, at 37° C.

12. The modified release softgel capsule of claim 1, wherein the modified release softgel capsule is annealed.

13. (canceled)

14. (canceled)

15. The modified release softgel capsule of claim 1, wherein the pH dependent shell composition further comprises a plasticizer.

16. (canceled)

17. (canceled)

18. The modified release softgel capsule of claim 1, wherein the pH dependent shell composition comprises about 25 wt. % to about 80 wt. % of a gelatin, based on the dry pH dependent shell composition weight.

19. The modified release softgel capsule of claim 1, wherein the pH dependent shell composition comprises about 2 wt. % to about 20 wt. % of pectin, based on the dry pH dependent shell composition weight.

20. The modified release softgel capsule of claim 1, wherein the pH dependent shell composition comprises about 0.005 wt. % to about 4 wt. % of dextrose, based on the dry pH dependent shell composition weight.

21.-28. (canceled)

29. The modified release softgel capsule of claim 1 that is free of additional pH dependent polymers.

30. The modified release softgel capsule of claim 1, wherein the pH dependent shell composition has a viscosity ranging from about 110,000 cPs to about 125,000 cPs.

31. The modified release softgel capsule of claim 1, wherein the pH dependent shell composition has a gelatin to pectin w:w ratio ranging from about 2:1 to about 20:1.

32. (canceled)

33. The modified release softgel capsule of claim 1, wherein the controlled release fill composition has a w:w ratio of the controlled release material to the at least one active agent ranging from about 10:1 to about 1:10.

34. The modified release softgel capsule of claim 1, wherein the at least one active agent is selected from pharmaceutically active ingredients, nutraceuticals, and a combination thereof.

35.-37. (canceled)

38. A process of preparing a modified release softgel capsule according to claim 1 comprising:
(a) preparing the controlled release fill composition; and
(b) encapsulating the controlled release fill composition with the pH dependent shell composition.

39. The process of claim 38, further comprising drying the encapsulated modified release softgel capsule.

40. The process of claim 38, further comprising annealing the modified release softgel capsule.

41.-46. (canceled)

47. A method for tuning the dissolution location and profile of a modified release softgel capsule comprised of a controlled release fill composition encapsulated in a pH dependent shell composition, the method comprising:
adjusting at least one of i)-iv) to control dissolution location of the pH dependent shell composition along the gastrointestinal tract of a subject:
i) amounts of at least one of pectin, gelatin, dextrose, and plasticizer in the pH dependent shell composition;
ii) annealing temperature of the modified release softgel capsule;
iii) annealing duration of the modified release softgel capsule; or
iv) ribbon thickness of the pH dependent shell composition; and
adjusting at least one of (v)-(vii) to attain a target dissolution profile of the at least one active agent:
v) amount of a controlled release material in the controlled release fill composition, or
vi) annealing temperature of the modified release softgel capsule; or
vii) annealing duration of the modified release softgel capsule.

48. A method of treating a condition comprising, administering to a subject in need thereof the modified release softgel capsule according to claim 1.

49. A modified release softgel capsule comprising:
(a) a controlled release fill composition comprising:
(i) at least one active agent; and
(ii) a polyethylene oxide having a number average molecular weight of from about 0.05M Dalton to about 15M Dalton; and
(iii) optionally a hydrophilic carrier; and
(b) a pH dependent shell composition encapsulating the controlled release fill composition, wherein the pH dependent shell composition comprises gelatin, pectin, dextrose, and optionally a plasticizer.

50. A method of preparing a modified release softgel capsule according to claim 49 comprising:
mixing at least one active agent with polyethylene oxide and optionally a hydrophilic carrier to form a controlled release fill composition;
encapsulating the controlled release fill composition in a pH dependent shell composition comprising gelatin, pectin, dextrose, and optionally a plasticizer; and
annealing the encapsulated controlled release fill composition.

51.-53. (canceled)

54. A modified release softgel capsule comprising:
(a) a controlled release fill composition comprising:
(i) at least one active agent; and
(ii) a controlled release material; and
(b) a pH dependent shell composition encapsulating the controlled release fill composition.

55.-71. (canceled)
